(19) United States
(12) Patent Application Publication
(10) Pub. No.: US 2025/0003974 A1
(43) Pub. Date: Jan. 2, 2025

(54) COMPOUNDS AND METHODS OF USE THEREOF FOR ASSESSING TARGET ENGAGEMENT FOR DNA POLYMERASE THETA

(71) Applicant: Promega Corporation, Madison, WI (US)

(72) Inventors: Michael Slater, Madison, WI (US); James Vasta, Madison, WI (US); Mathew Robers, Madison, WI (US); Matthew Larsen, Madison, WI (US); Cesear Corona, Madison, WI (US); Weiwei An, Madison, WI (US); Min Zhou, Madison, WI (US); Wenhui Zhou, Madison, WI (US)

(21) Appl. No.: 18/749,789

(22) Filed: Jun. 21, 2024

Related U.S. Application Data
(60) Provisional application No. 63/509,704, filed on Jun. 22, 2023.

(51) Int. Cl. G01N 33/58; C07F 5/02; C12N 9/12; G01N 33/573

(52) U.S. Cl. CPC G01N 33/582; C07F 5/022; C12N 9/1252; C12Y 207/07007; G01N 33/573; C07K 2319/61; G01N 2333/9126

(57) ABSTRACT

The present invention provides compounds and methods of use thereof for assessing target engagement for DNA polymerase theta. In particular, provided herein are tracer compounds comprising a small molecule that binds DNA polymerase theta tethered to a fluorophore, and methods of use thereof in target engagement assays.

### PRIORITY STATEMENT

This application claims priority to U.S. Provisional Application No. 63/509,704, filed Jun. 22, 2023, the entire contents of which are incorporated herein by reference for all purposes.

### SEQUENCE LISTING

The text of the computer readable sequence listing filed herewith, titled “PRMG-42123-202_SQL”, created Jun. 22, 2023, having a file size of 7,137 bytes, is hereby incorporated by reference in its entirety.

### FIELD

The present invention provides compounds and methods of use thereof for assessing target engagement for DNA polymerase theta. In particular, provided herein are tracer compounds comprising a small molecule that binds DNA polymerase theta tethered to a fluorophore, and methods of use thereof in target engagement assays.

### BACKGROUND

DNA polymerase theta (PolΘ) represents a critical drug target in the DNA damage response (DDR) pathway owing to its role in synthetic lethality in homologous repair-deficient tumors. It is also targeted in poly (ADP-ribose) polymerase (PARP)-inhibitor resistant cells, compounding its value in drug discovery. However, there are no direct methods to query drug target engagement at polymerase theta or biomolecular complexes of polymerase theta and damaged DNA fragments in cells, representing a critical shortcoming for drug hunters. Accordingly, what is needed are compositions and methods for assessing target engagement for polymerase theta in the presence and absence of damaged DNA fragments.

### SUMMARY

In some aspects, provided herein are tracer compounds of formula (Ia) or (Ib):

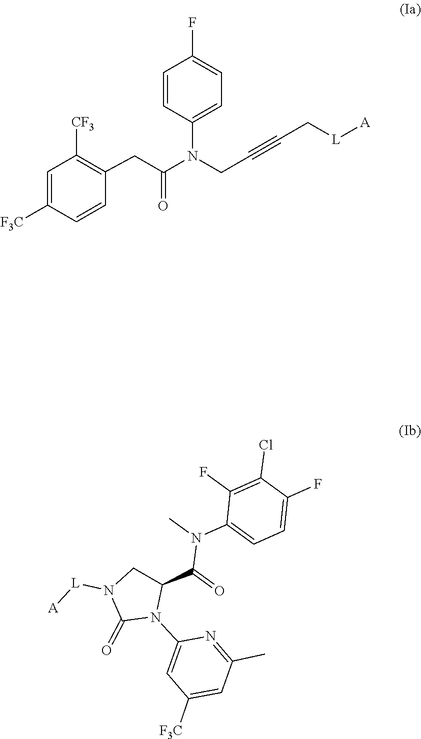

or a salt thereof, wherein:
L is a linker; and
A is an energy transfer acceptor.

In some embodiments, L has a formula:

—NH—(CH₂CH₂O)ₙ₁—(CH₂)ₙ₂—(C(O)NH)ₙ₃—(CH₂)ₙ₄—
wherein n1, n2, and n4 are each independently selected from 0, 1, 2, 3, 4, 5, and 6, and n3 is 0 or 1.

In some embodiments, L has a formula:

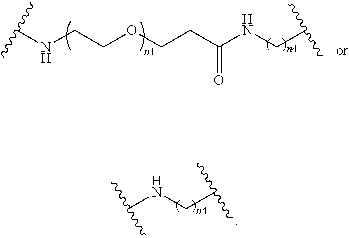

In some embodiments, A is a fluorophore selected from a xanthene, a cyanine, a naphthalene, an oxadiazole, a pyrene, an oxazine, an acridine, an arylmethine, a tetrapyrrole, and a boron dipyrromethene.

In some embodiments, A is a BODIPY dye.

In some embodiments, A has a formula:

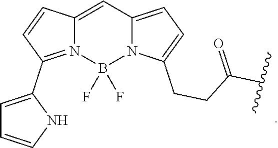

In some embodiments, the tracer compound is selected from:

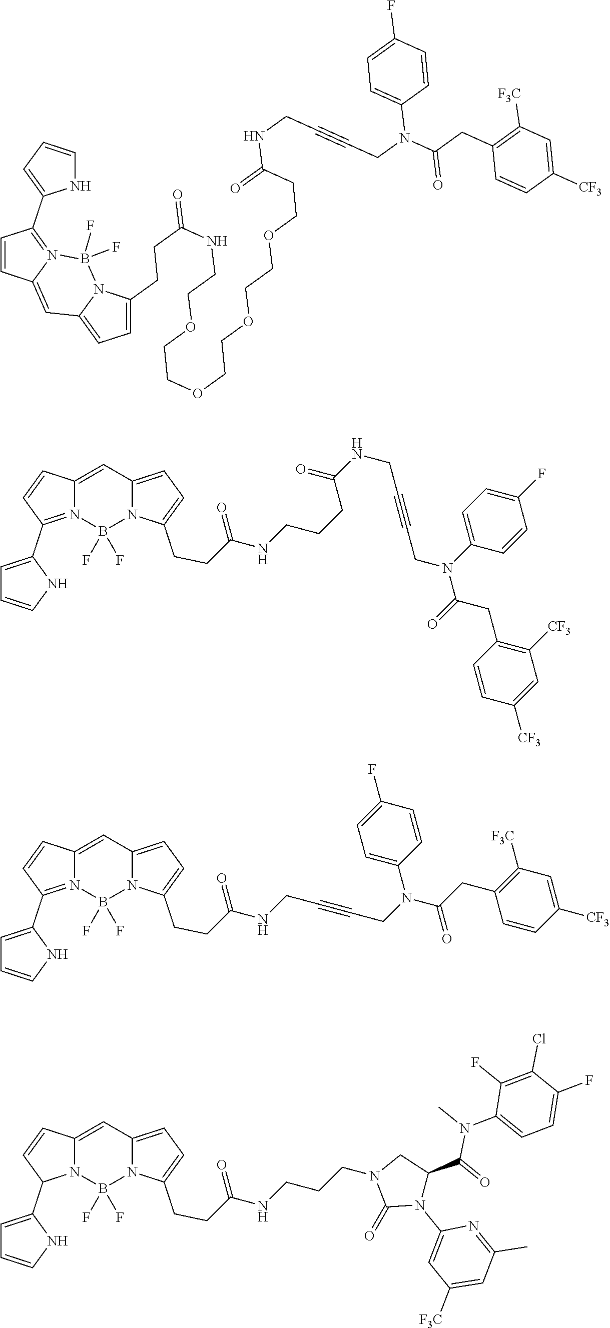

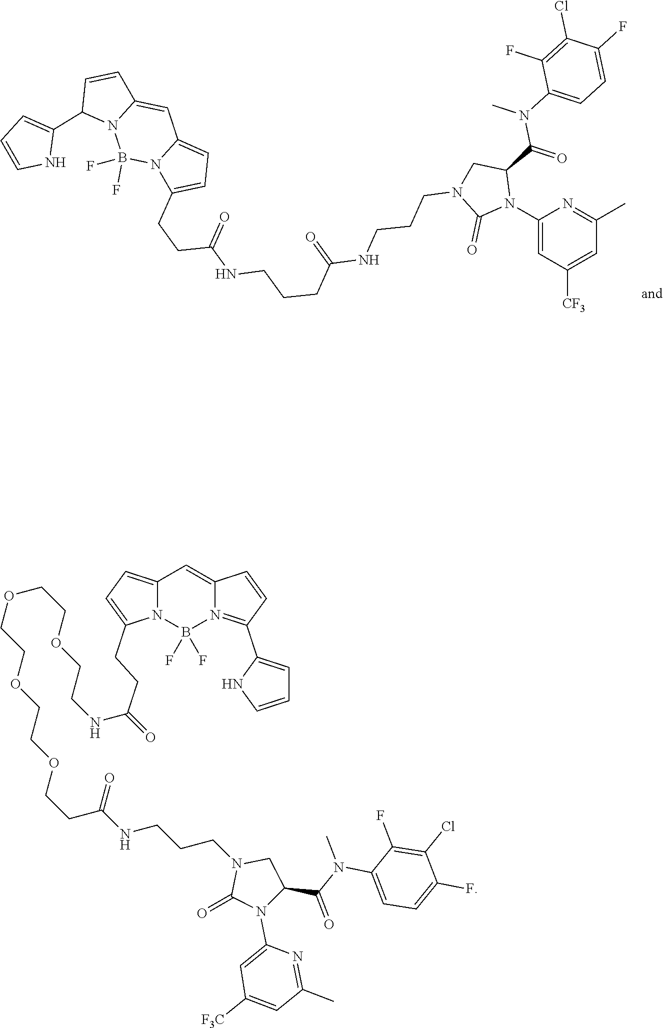

In some aspects, provided herein are tracer compounds of formula (II):

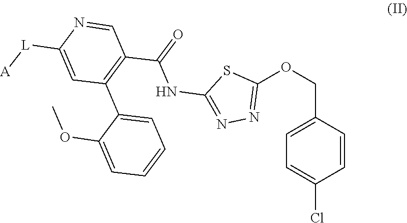

or a salt thereof, wherein:
L is a linker; and
A is an energy transfer acceptor.

In some embodiments, L has a formula:

—NH—(CH₂CH₂O)ₙ₁—(CH₂)ₙ₂—(C(O)NH)ₙ₃—(CH₂)ₙ₄—
wherein n1, n2, and n4 are each independently selected from 0, 1, 2, 3, 4, 5, and 6, and n3 is 0 or 1.

In some embodiments, L has a formula:

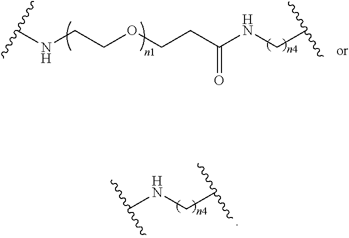

In some embodiments, A is a fluorophore selected from a xanthene, a cyanine, a naphthalene, an oxadiazole, a pyrene, an oxazine, an acridine, an arylmethine, a tetrapyrrole, and a boron dipyrromethene.

In some embodiments, A is a BODIPY dye.

In some embodiments, A has a formula:

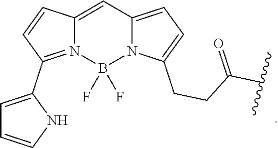

In some embodiments, the tracer compound is selected from:

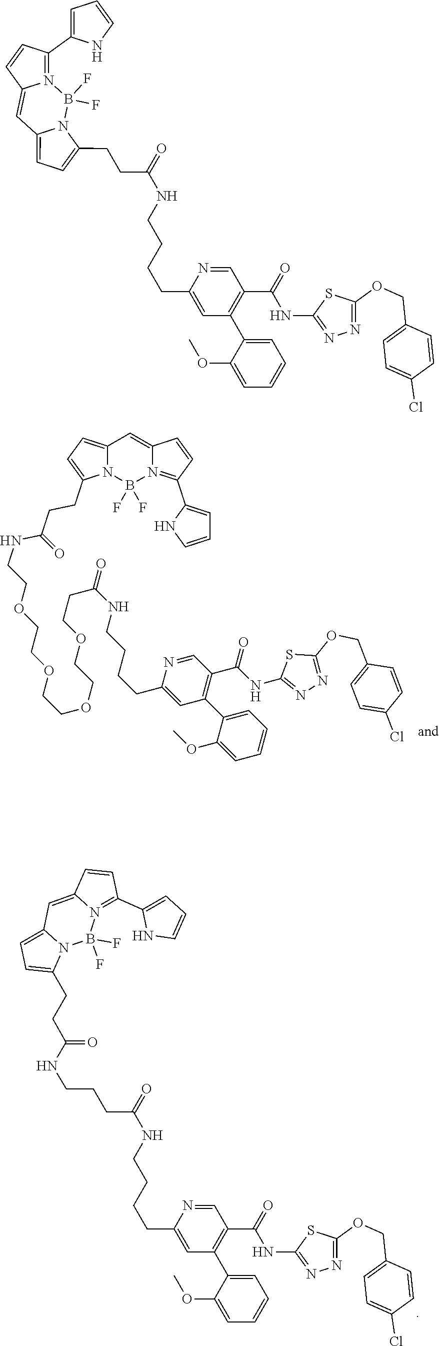

In some aspects, provided herein are fusion proteins comprising a bioluminescent reporter and DNA polymerase theta (Polθ). In some embodiments, the bioluminescent reporter comprises a luciferase. For example, in some embodiments the luciferase comprises a polypeptide with at least 70% sequence identity with SEQ ID NO.: 1.

In some aspects, provided herein are systems comprising a cell that expresses a fusion protein comprising a bioluminescent reporter and DNA polymerase theta (Polθ); one or more DNA fragments; a tracer compound of formula Ia or formula Ib; and a substrate for the bioluminescent reporter. In some embodiments, the tracer compound binds coordinatively with the one or more DNA fragments to the polymerase domain of Polθ. In some embodiments, upon coordinative binding of the tracer compound and the one or more DNA fragments to the polymerase domain of Polθ, conversion of the substrate to a reaction product by the bioluminescent reporter results in BRET, which is measured by increased emission from the fluorophore. In some embodiments, the one or more DNA fragments each form a hairpin structure with a single-stranded overhang. In some embodiments, the single-stranded overhang comprises 5-30 bases.

In some aspects, provided herein are system comprising a cell that expresses a fusion protein comprising a bioluminescent reporter and DNA polymerase theta (Polθ); a tracer compound of formula II, and a substrate for the bioluminescent reporter. In some embodiments, the tracer compound binds to the helicase domain of Polθ. In some embodiments, upon binding of the tracer compound to the helicase domain of Polθ, conversion of the substrate to a reaction product by the bioluminescent reporter results in BRET, which is measured by increased emission from the fluorophore.

In some embodiments, the emission spectrum of the bioluminescent reporter overlaps with the excitation spectrum of the fluorophore. In some embodiments, the bioluminescent reporter has a first emission spectrum with a first peak emission, the fluorophore has an excitation spectrum that overlaps said first emission spectrum, and the fluorophore has a second emission spectrum with a second peak emission, the second peak emission being substantially separated from said first peak emission. In some embodiments, intensity of fluorescence within the second emission spectrum correlates with binding between the tracer compound and Polθ.

Any of the system described herein may further comprise a test compound. In some embodiments, the test compound is a putative engager of Polθ. In some embodiments, the systems provided herein are used in a method of detecting an interaction between a test compound and Polθ. For example, in some embodiments the system further comprises a test compound, and if the test compound binds to Polθ the BRET signal from the system is reduced upon binding.

In some aspects, provided herein are methods of detecting an interaction between a test compound and DNA polymerase theta (Polθ). In some embodiments, the method comprises providing a cell that expresses a fusion protein comprising a bioluminescent reporter and DNA polymerase theta (Polθ), contacting the cell with one or more DNA fragments, a substrate for the bioluminescent reporter, and a tracer compound of formula Ia or formula Ib, and detecting a change in BRET that occurs upon adding contacting the cell with a test compound. In some embodiments, the tracer compound binds coordinatively with the one or more DNA fragments to Polθ. In some embodiments, the method comprises detecting a first BRET signal prior to contacting the cell with the test compound, wherein the first BRET signal is indicative of coordinative binding of the tracer compound and the one or more DNA fragments to Polθ. In some embodiments, the method comprises detecting a second BRET signal after contacting the cell with the test compound. In some embodiments, a decrease in the second BRET signal from the first BRET signal indicates that the test compound is an engager of Polθ. In some embodiments, the tracer compound binds uncompetitively with the one or more DNA fragments to the polymerase domain of Polθ, and a decrease in the second BRET signal from the first BRET signal indicates that the test compound is an engager of the polymerase domain of Polθ.

In some aspects, provided herein are methods of detecting an interaction between a test compound and DNA polymerase theta (Polθ), comprising providing a cell that expresses a fusion protein comprising a bioluminescent reporter and DNA polymerase theta (Polθ), contacting the cell with a substrate for the bioluminescent reporter, and a tracer compound of formula (II), and detecting a change in BRET that occurs upon contacting the cell with a test compound. In some embodiments, the method comprises obtaining a first BRET signal prior to contacting the cell with the test compound, wherein the first BRET signal is indicative of binding of the tracer compound to Polθ. In some embodiments, the method comprises detecting a second BRET signal after contacting the cell with the test compound. In some embodiments, a decrease in the second BRET signal from the first BRET signal indicates that the test compound is an engager of Polθ. In some embodiments, the tracer compound binds to the helicase domain of Polθ, and a decrease in the second BRET signal from the first BRET signal indicates that the test compound is an engager of the helicase domain of Polθ.

In some embodiments, the bioluminescent reporter comprises a luciferase. For example, in some embodiments the luciferase comprises a polypeptide with at least 70% sequence identity with SEQ ID NO: 1. In some embodiments, the substrate for the luciferase is coelenterazine or a coelenterazine derivative.

### DRAWINGS

FIG. 1A shows BRET measurements in permeabilized cells expressing POLQ/NanoLuc (Nluc) fusions. Cells were permeabilized with digitonin and contacted with various exemplary tracers with and without added DNA fragments. For each tracer, addition of DNA fragments potentiated BRET signal. FIG. 1B shows a schematic of experiments conducted in permeabilized cells contacted with the tracer 9625 and the control polymerase theta inhibitor 9618 (“unmodified compound”), with and without added DNA fragments. Graphs showing BRET measurements are shown in the bottom panel.

FIG. 2 shows BRET measurements in live cells expressing POLQ/NanoLuc (Nluc) fusions following delivery of DNA fragments by electroporation and treatment with an exemplary tracer 9686 and the control polymerase theta inhibitor 9618.

FIG. 3 shows a schematic of experiments conducted in live cells expressing POLQ/NanoLuc (Nluc) fusion contacted with the tracer 9686 and the control polymerase theta inhibitor 9618, with and without DNA fragments delivered by electroporation. Graphs showing BRET measurements are shown in the bottom panel.

FIGS. 4A-4B are graphs evaluating the effect of differing DNA fragments on BRET transfer. FIG. 4A shows BRET measurements in live cells expressing POLQ/NanoLuc (Nluc) fusion contacted with tracer 9686 and the control polymerase theta 9618 and electroporated with “lanky” or “bulky” DNA fragments. FIG. 4B shows graphs showing BRET measurements in live cells expressing POLQ/NanoLuc (Nluc) fusion contacted with various tracers and varying concentrations of “lanky” and “stubby” DNA fragments.

FIG. 5 shows BRET measurements in live cells expressing NanoLuc (Nluc)-POLQ fusions contacted with the tracer 9847 and the control inhibitor 9617, each of which target the helicase domain of polymerase theta. BRET measurements were performed with and without delivery of DNA fragments. As seen in FIG. 5, a suitable NanoBRET signal with the tracer was observed without added DNA fragments.

FIG. 6 shows exemplary tracers provided herein that target the polymerase domain of DNA polymerase theta.

FIG. 7 shows an exemplary control inhibitor 9618 which targets the polymerase domain of DNA polymerase theta.

FIG. 8 show exemplary DNA fragments developed and tested herein.

FIG. 9 shows exemplary tracers provided herein that target the helicase domain of DNA polymerase theta.

FIG. 10 shows an exemplary control inhibitor 9617 which targets the helicase domain of DNA polymerase theta.

FIG. 11 show BRET measurements live or permeabilized cells expressing POLQ-NanoLuc (Nluc) fusion transfected with varying amounts of “stubby” oligo DNA fragments and treated with the tracer 9686 and the control inhibitor 9618 or DMSO. BRET measurements are shown in the top panel. The bottom panel shows enhanced target engagement of the control inhibitor 9618 in the presence of DNA fragment.

### DEFINITIONS

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies, or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a,” “an,” and “the” include plural reference unless the context clearly dictates otherwise. Thus, for example, reference to “a polypeptide” is a reference to one or more polypeptides and equivalents thereof known to those skilled in the art, and so forth.

As used herein, the term “and/or” includes any and all combinations of listed items, including any of the listed items individually. For example, “A, B, and/or C” encompasses A, B, C, AB, AC, BC, and ABC, each of which is to be considered separately described by the statement “A, B, and/or C.”

As used herein, the term “comprise” and linguistic variations thereof denote the presence of recited feature(s), element(s), method step(s), etc., without the exclusion of the presence of additional feature(s), element(s), method step(s), etc. Conversely, the term “consisting of” and linguistic variations thereof, denotes the presence of recited feature(s), element(s), method step(s), etc., and excludes any unrecited feature(s), element(s), method step(s), etc., except for ordinarily-associated impurities. The phrase “consisting essentially of” denotes the recited feature(s), element(s), method step(s), etc., and any additional feature(s), element(s), method step(s), etc., that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

As used herein, the term “system” refers to multiple components (e.g., devices, compositions, etc.) that find use for a particular purpose. For example, two separate biological molecules, whether present in the same composition or not, may comprise a system if they are useful together for a shared purpose.

As used herein, the term “complementary” refers to the characteristic of two or more structural elements (e.g., peptide, polypeptide, nucleic acid, small molecule, etc.) of being able to hybridize, dimerize, or otherwise form a complex with each other. For example, a “complementary peptide and polypeptide” are capable of coming together to form a complex. Complementary elements may require assistance (facilitation) to form a complex (e.g., from interaction elements), for example, to place the elements in the proper conformation for complementarity, to place the elements in the proper proximity for complementarity, to co-localize complementary elements, to lower interaction energy for complementary, to overcome insufficient affinity for one another, etc.

The term “amino acid” refers to natural amino acids, unnatural amino acids, and amino acid analogs, all in their D and L stereoisomers, unless otherwise indicated, if their structures allow such stereoisomeric forms.

As used herein, unless otherwise specified, the terms “peptide” and “polypeptide” refer to polymer compounds of two or more amino acids joined through the main chain by peptide amide bonds (—C(O)NH—). The term “peptide” typically refers to short amino acid polymers (e.g., chains having fewer than 30 amino acids), whereas the term “polypeptide” typically refers to longer amino acid polymers (e.g., chains having more than 30 amino acids).

As used herein, a “conservative” amino acid substitution refers to the substitution of an amino acid in a peptide or polypeptide with another amino acid having similar chemical properties such as size or charge. For purposes of the present disclosure, each of the following eight groups contains amino acids that are conservative substitutions for one another:
1) Alanine (A) and Glycine (G);
2) Aspartic acid (D) and Glutamic acid (E);
3) Asparagine (N) and Glutamine (Q);
4) Arginine (R) and Lysine (K);
5) Isoleucine (I), Leucine (L), Methionine (M), and Valine (V);
6) Phenylalanine (F), Tyrosine (Y), and Tryptophan (W);
7) Serine (S) and Threonine (T); and
8) Cysteine (C) and Methionine (M).

Amino acid residues may be divided into classes based on common side chain properties, for example: polar positive (or basic) (e.g., histidine (H), lysine (K), and arginine (R)); polar negative (or acidic) (e.g., aspartic acid (D), glutamic acid (E)); polar neutral (e.g., serine (S), threonine (T), asparagine (N), glutamine (Q)); non-polar aliphatic (e.g., alanine (A), valine (V), leucine (L), isoleucine (I), methionine (M)); non-polar aromatic (e.g., phenylalanine (F), tyrosine (Y), tryptophan (W)); proline and glycine; and cysteine. As used herein, a “semi-conservative” amino acid substitution refers to the substitution of an amino acid in a peptide or polypeptide with another amino acid within the same class.

In some embodiments, unless otherwise specified, a conservative or semi-conservative amino acid substitution may also encompass non-naturally occurring amino acid residues that have similar chemical properties to the natural residue. These non-natural residues are typically incorporated by chemical peptide synthesis rather than by synthesis in biological systems. These include, but are not limited to, peptidomimetics and other reversed or inverted forms of amino acid moieties. Embodiments herein may, in some embodiments, be limited to natural amino acids, non-natural amino acids, and/or amino acid analogs.

Non-conservative substitutions may involve the exchange of a member of one class for a member from another class.

As used herein, the term “sequence identity” refers to the degree two polymer sequences (e.g., peptide, polypeptide, nucleic acid, etc.) have the same sequential composition of monomer subunits. The term “sequence similarity” refers to the degree with which two polymer sequences (e.g., peptide, polypeptide, nucleic acid, etc.) have similar polymer sequences. For example, similar amino acids are those that share the same biophysical characteristics and can be grouped into the families, e.g., acidic (e.g., aspartate, glutamate), basic (e.g., lysine, arginine, histidine), non-polar (e.g., alanine, valine, leucine, isoleucine, proline, phenylalanine, methionine, tryptophan) and uncharged polar (e.g., glycine, asparagine, glutamine, cysteine, serine, threonine, tyrosine). The “percent sequence identity” (or “percent sequence similarity”) is calculated by: (1) comparing two optimally aligned sequences over a window of comparison (e.g., the length of the longer sequence, the length of the shorter sequence, a specified window), (2) determining the number of positions containing identical (or similar) monomers (e.g., same amino acids occurs in both sequences, similar amino acid occurs in both sequences) to yield the number of matched positions, (3) dividing the number of matched positions by the total number of positions in the comparison window (e.g., the length of the longer sequence, the length of the shorter sequence, a specified window), and (4) multiplying the result by 100 to yield the percent sequence identity or percent sequence similarity. For example, if peptides A and B are both 20 amino acids in length and have identical amino acids at all but 1 position, then peptide A and peptide B have 95% sequence identity. If the amino acids at the non-identical position shared the same biophysical characteristics (e.g., both were acidic), then peptide A and peptide B would have 100% sequence similarity. As another example, if peptide C is 20 amino acids in length and peptide D is 15 amino acids in length, and 14 out of 15 amino acids in peptide D are identical to those of a portion of peptide C, then peptides C and D have 70% sequence identity, but peptide D has 93.3% sequence identity to an optimal comparison window of peptide C. For the purpose of calculating “percent sequence identity” (or “percent sequence similarity”) herein, any gaps in aligned sequences are treated as mismatches at that position.

Any peptide/polypeptides described herein as having a particular percent sequence identity or similarity (e.g., at least 70%) with a reference sequence ID number, may also be expressed as having a maximum number of substitutions (or terminal deletions) with respect to that reference sequence. For example, a sequence having at least Y % sequence identity (e.g., 90%) with SEQ ID NO:Z (e.g., 100 amino acids) may have up to X substitutions (e.g., 10) relative to SEQ ID NO:Z, and may therefore also be expressed as “having X (e.g., 10) or fewer substitutions relative to SEQ ID NO:Z.”

As used herein, the terms “energy acceptor” and “energy transfer acceptor” are used interchangeably herein and refer to any small molecule (e.g., chromophore), macromolecule (e.g., autofluorescent protein, phycobiliproteins, nanoparticle, surface, etc.), or molecular complex that produces a readily detectable signal in response to energy absorption (e.g., resonance energy transfer). In certain embodiments, an energy transfer acceptor is a fluorophore or other detectable chromophore.

As used herein, the terms “fusion,” “fusion polypeptide,” and “fusion protein” refer to a chimeric protein containing a first protein or polypeptide of interest joined to a second different peptide, polypeptide, or protein.

As used herein, the terms “conjugated” and “conjugation” refer to the covalent attachment of two molecular entities (e.g., post-synthesis and/or during synthetic production). The attachment of a peptide or small molecule tag to a protein or small molecule, chemically (e.g., “chemically” conjugated) or enzymatically, is an example of conjugation.

As used herein, the term “bioluminescence” refers to production and emission of light by a chemical reaction catalyzed by, or enabled by, an enzyme, protein, protein complex, or other biomolecule (e.g., bioluminescent complex). In typical embodiments, a substrate for a bioluminescent entity (e.g., bioluminescent protein or bioluminescent complex) is converted into an unstable form by the bioluminescent entity; the substrate subsequently emits light.

As used herein, the term “an Oplophorus luciferase” (“an OgLuc”) refers to a luminescent polypeptide having significant sequence identity, structural conservation, and/or the functional activity of the luciferase produce by and derived from the deep-sea shrimp Oplophorus gracilirostris. In particular, an OgLuc polypeptide refers to a luminescent polypeptide having significant sequence identity, structural conservation, and/or the functional activity of the mature 19 kDa subunit of the Oplophorus luciferase protein complex (e.g., without a signal sequence) such as SEQ ID NOs: 1 (NanoLuc/NLuc), which comprises 10 β strands (β1, β2, β3, β4, β5, β6, β7, β8, β9, β10) and utilize substrates such as coelenterazine or a coelenterazine derivative or analog to produce luminescence.

As used herein, the term “NANOLUC” or “NLUC” refers to an artificial luciferase or bioluminescent polypeptide produced commercially by the Promega Corporation.

### DETAILED DESCRIPTION

The present invention provides tracers, fusion proteins, DNA fragments, BRET assay systems, and methods for detection and analysis of target engagement with DNA polymerase theta. In some aspects, provided herein are compounds tethered to a fluorophore, fusion proteins comprising a bioluminescent reporter protein fused to a DNA polymerase theta (PolΘ), and systems comprising the same for use in methods of analyzing the interaction of agents with DNA polymerase theta.

### Compounds

In some aspects, provided herein are tracer compounds of formula (Ia) and formula (Ib):

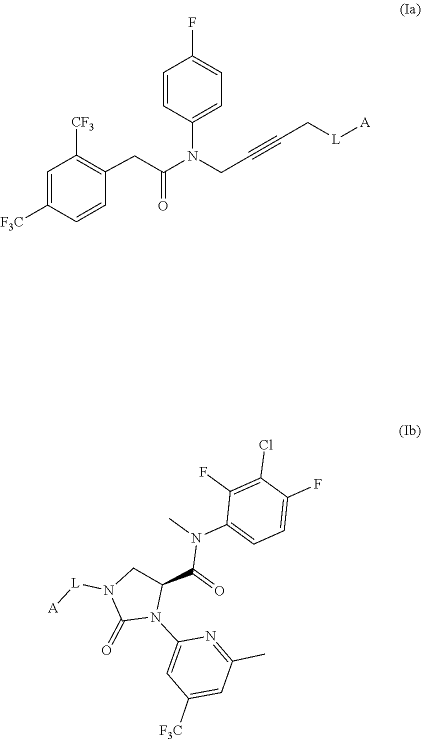

and salts thereof, wherein:
L is a linker; and
A is an energy transfer acceptor.

In some aspects, provided herein are tracer compounds of formula (II):

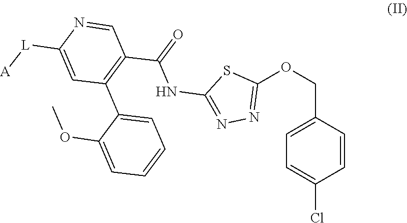

and salts thereof, wherein:
L is a linker; and
A is an energy transfer acceptor.

The tracer compounds include an energy transfer acceptor. In some embodiments, an energy transfer acceptor is an entity capable of generating, exhibiting, and/or emitting a signal (e.g., light, heat, chemical reaction, fluorescence, resonance energy, etc.) when triggered by energy absorption (e.g., resonance energy transfer). In some embodiments, the energy transfer acceptor is a chromophore (e.g., fluorophore). Compounds tethered to an energy transfer acceptor (e.g., fluorophore) are referred to herein as a tracer or a fluorescent tracer.

In some embodiments, the energy transfer acceptor is a fluorophore. Suitable fluorophores include, but are not limited to: xanthene derivatives (e.g., fluorescein, rhodamine, Oregon green, eosin, Texas red, etc.), cyanine derivatives (e.g., cyanine, indocarbocyanine, oxacarbocyanine, thiacarbocyanine, merocyanine, etc.), naphthalene derivatives (e.g., dansyl and prodan derivatives), oxadiazole derivatives (e.g., pyridyloxazole, nitrobenzoxadiazole, benzoxadiazole, etc.), pyrene derivatives (e.g., cascade blue), oxazine derivatives (e.g., Nile red, Nile blue, cresyl violet, oxazine 170, etc.), acridine derivatives (e.g., proflavin, acridine orange, acridine yellow, etc.), arylmethine derivatives (e.g., auramine, crystal violet, malachite green, etc.), tetrapyrrole derivatives (e.g., porphin, phtalocyanine, bilirubin, etc.), CF dye (Biotium), BODIPY (Invitrogen), ALEXA FLuoR (Invitrogen), DYLIGHT FLUOR (Thermo Scientific, Pierce), ATTO and TRACY (Sigma Aldrich), FluoProbes (Interchim), DY and MEGASTOKES (Dyomics), SULFO CY dyes (CYANDYE, LLC), SETAU AND SQUARE DYES (SETA BioMedicals), QUASAR and CAL FLUOR dyes (Biosearch Technologies), SURELIGHT DYES (APC, RPE, PerCP, Phycobilisomes)(Columbia Biosciences), APC, APCXL, RPE, BPE (Phyco-Biotech), autofluorescent proteins (e.g., YFP, RFP, mCherry, mKate), quantum dot nanocrystals, etc. In some embodiments, a fluorophore is a rhodamine analog (e.g., carboxy rhodamine analog), such as those described in U.S. patent application Ser. No. 13/682,589, herein incorporated by reference in its entirety. In some embodiments, BRET efficiency is significantly enhanced by the technical features of rhodamine analog (e.g., carboxy rhodamine analog) as an energy transfer acceptor compared to other fluorophores. For example, the left shifted EC50 and reduced nonspecific background of these dyes is advantageous for use in some embodiments. In some embodiments, the fluorophore is a BODIPY dye. For example, in some embodiments, A has a formula:

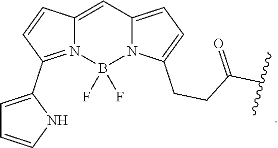

The tracer compounds also include a linker, which serves to link the energy transfer acceptor to the remainder of the compound. In some embodiments, the linker provides sufficient distance between the energy transfer acceptor and the rest of the compound, to allow each to function undisturbed (or minimally disturbed) by the linkage to the other. For example, in embodiments in which the energy transfer acceptor is a fluorophore, the linker provides sufficient distance to allow the tracer compound to bind to DNA polymerase theta and also to allow the fluorophore to be detectable (e.g., without interference or with minimal interference). In some embodiments, the linker separates the energy transfer acceptor (e.g., fluorophore) from the rest of the compound by 5 Å to 1000 Å, inclusive, in length. In some embodiments, the linker separates the functional element from the rest of the compound of formula (I) by 5 Å, 10 Å, 20 Å, 50 Å, 100 Å, 150 Å, 200 Å, 300 Å, 400 Å, 500 Å, 600 Å, 700 Å, 800 Å, 900 Å, 1000 Å, or any suitable range therebetween (e.g., 5-100 Å, 50-500 Å, 150-700 Å, etc.). In some embodiments, the linker separates the energy transfer acceptor from the rest of the compound by 1-200 atoms (e.g., 1, 2, 3, 4, 5, 6, 7, 8, 9, 10, 15, 20, 25, 30, 35, 40, 45, 50, 60, 70, 80, 90, 100, 120, 140, 160, 180, 200, or any suitable ranges therebetween (e.g., 2-20, 10-50, etc.)).

The linker can include one or more groups independently selected from methylene (—CH₂—), ether (—O—), amine (—NH—), alkylamine (—NR—), wherein R is an optionally substituted C₁-C₆ (alkyl group), thioether (—S—), disulfide (—S—S—), amide (—C(O)NH—), ester (—C(O)O—), carbamate (—OC(O)NH—), sulfonamide (—S(O)₂NH—), phenylene (—C₆H₄—), and piperazinylene

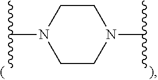 [SMILES: *N1CCN(*)CC1]

and any combination thereof.

In some embodiments, the linker comprises one or more —(CH₂CH₂O)— (oxyethylene) groups, e.g., 1-20 —(CH₂CH₂O)— groups (e.g., 1, 2, 3, 4, 5, 6, 7, 8, 9, 10, 11, 12, 13, 14, 15, 16, 17, 18, 19, or 20 —(CH₂CH₂O)— groups, or any range therebetween). In some embodiments, the linker comprises a —(CH₂CH₂O)—, —(CH₂CH₂O)₂—, —(CH₂CH₂O)₃—, —(CH₂CH₂O)₄—, —(CH₂CH₂O)₅—, —(CH₂CH₂O)₆—, —(CH₂CH₂O)₇—, —(CH₂CH₂O)₈—, —(CH₂CH₂O)₉—, or —(CH₂CH₂O)₁₀— group. In some embodiments, the linker comprises a —(CH₂CH₂O)₄— group.

In some embodiments, the linker comprises one or more alkylene groups (e.g., —(CH₂)ₙ—, wherein n is 1-12, e.g., 1, 2, 3, 4, 5, 6, 7, 8, 9, 10, 11, or 12, or any suitable range therebetween). In some embodiments, the linker comprises one or more branched alkylene groups.

In some embodiments, the linker comprises at least one amide group (—C(O)NH—). In some embodiments, the linker comprises two amide groups.

In some embodiments, the linker comprises at least one piperazinylene group.

In some embodiments, the linker comprises a cleavable (e.g., enzymatically cleavable, chemically cleavable, etc.) moiety.

In some embodiments, the linker comprises one or more substituents, pendants, side chains, etc., comprising any suitable organic functional groups (e.g., —OH, —NH₂, —SH, —CN, ═O, ═S, halogen (e.g., —F, —Cl, —Br, —I), —COOH, —CONH₂, —CH₃, etc.).

In some embodiments, the linker comprises more than one linearly connected C, S, N, and/or O atoms. In some embodiments, the linker comprises 1-200 linearly connected atoms (e.g., 1, 2, 3, 4, 5, 6, 7, 8, 9, 10, 15, 20, 25, 30, 35, 40, 45, 50, 60, 70, 80, 90, 100, 120, 140, 160, 180, 200, or any suitable ranges therebetween (e.g., 2-20, 10-50, 6-18)). In some embodiments, the linker comprises 1-200 linearly connected atoms (e.g., 1, 2, 3, 4, 5, 6, 7, 8, 9, 10, 15, 20, 25, 30, 35, 40, 45, 50, 60, 70, 80, 90, 100, 120, 140, 160, 180, 200, or any suitable ranges therein (e.g., 2-20, 10-50, 6-18)).

In some embodiments, the linker L has formula:

—NH—(CH₂CH₂O)ₙ₁—(CH₂)ₙ₂—(C(O)NH)ₙ₃—(CH₂)ₙ₄—
wherein n1, n2, and n4 are each independently selected from 0, 1, 2, 3, 4, 5, and 6, and n3 is 0 or 1. In some embodiments, n1 is 0. In some embodiments, n1 is 4. In some embodiments, n2 is 0. In some embodiments, n2 is 2. In some embodiments, n3 is 0. In some embodiments, n3 is 1. In some embodiments, n4 is 0. In some embodiments, n4 is 3. In some embodiments, n4 is 4.

In some embodiments, the linker L has a formula selected from:

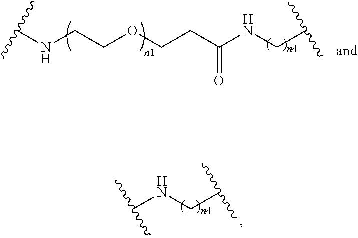

wherein n1 is 0, 1, 2, 3, or 4, and n4 is 0, 1, 2, 3, or 4. In some embodiments, n1 is 0. In some embodiments, n1 is 4. In some embodiments, n4 is 0. In some embodiments, n4 is 3. In some embodiments, n4 is 4.

In some embodiments, the linker has a formula selected from:

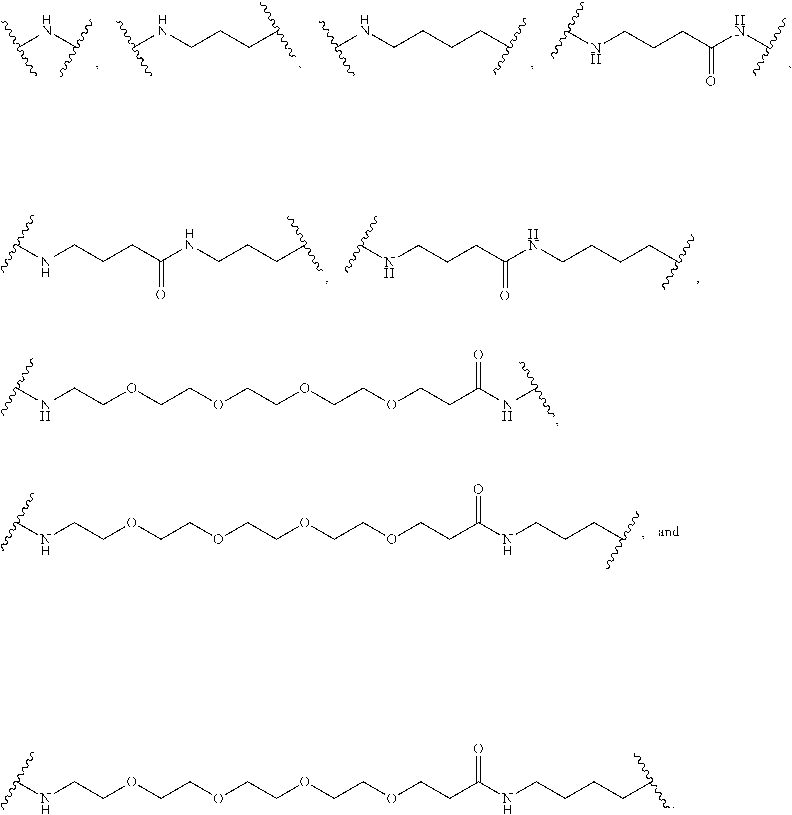

In some embodiments, the tracer compound is a compound of formula (Ia), selected from:

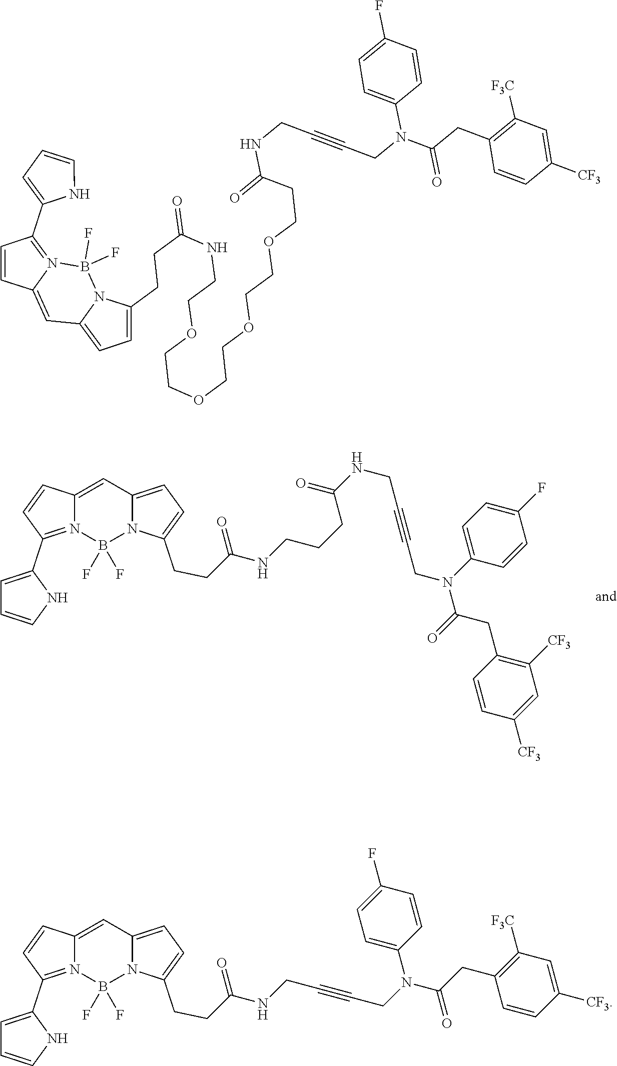

In some embodiments, the tracer compound is a compound of formula (Ib), selected from:

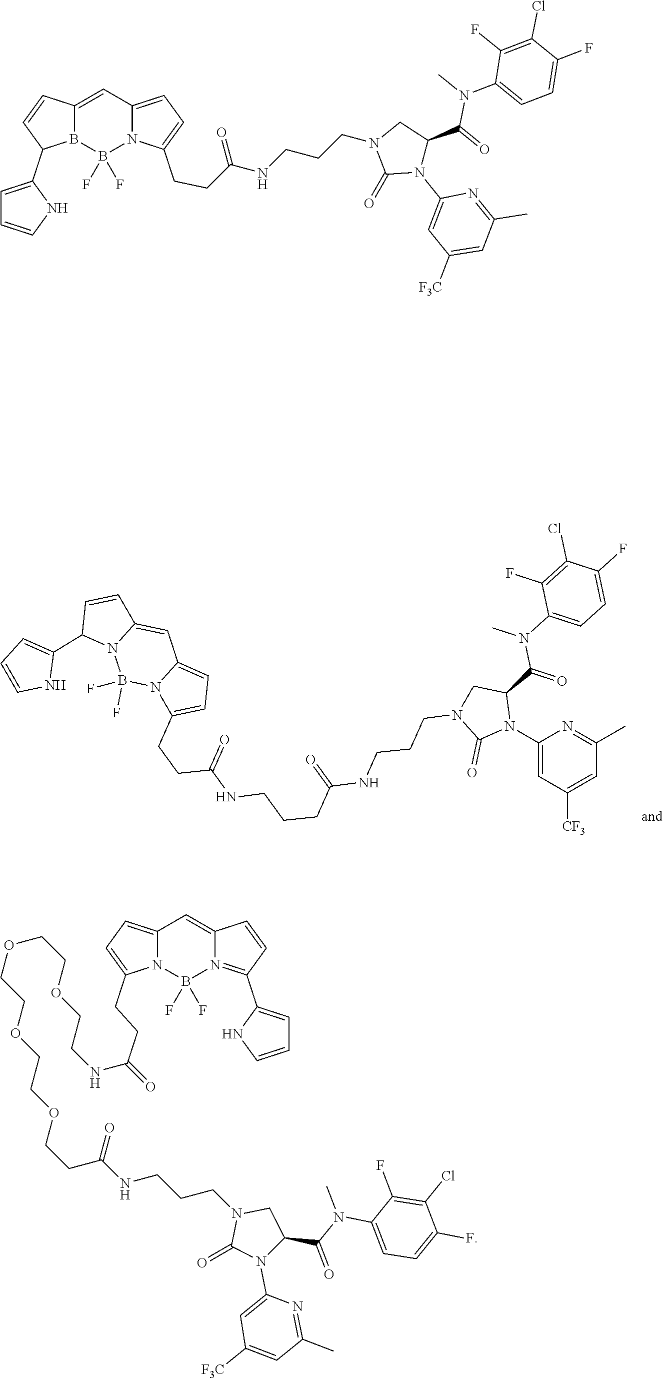

In some embodiments, the tracer compound is a compound of formula (II), selected from:

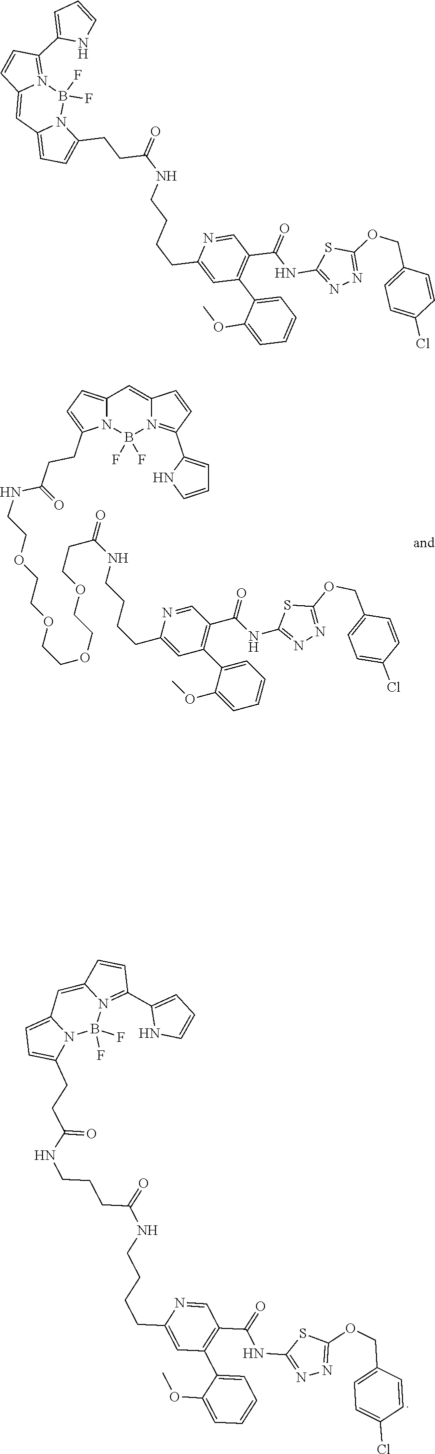

### Fusion Proteins

In some aspects, provided herein are fusion proteins comprising a bioluminescent reporter (e.g., a bioluminescent reporter protein) and DNA polymerase theta. The term “bioluminescent reporter protein” refers to a protein that can serve as an energy donor for transfer of energy from the reporter protein to an energy acceptor (e.g., fluorescence, light energy, resonance, such as by bioluminescence resonance energy transfer (BRET)). In some embodiments, the bioluminescent reporter protein is a luciferase. In some embodiments, a luciferase is selected from those found in Gaussia, coleoptera, (e.g., fireflies), Renilla, Vargula, Oplophorus, Aequoria, mutants thereof, portions thereof, variants thereof, and any other luciferase enzymes suitable for the systems and methods described herein. In some embodiments, the bioluminescent reporter protein is a modified, enhanced luciferase enzyme from Oplophorus (e.g., NANOLUC enzyme from Promega Corporation, SEQ ID NO: 1, or a sequence with at least 70% identity (e.g., >70%, >80%, >90%, >95%) thereto). In some embodiments, the bioluminescent reporter protein is a thermostable Photuris pennsylvanica luciferase or a sequence with at least 70% identity (e.g., >70%, >80%, >90%, >95%) thereto). Exemplary bioluminescent reporters are described, for example, in U.S. Pat. App. No. 2010/0281552 and U.S. Pat. App. No. 2012/0174242, both of which are herein incorporated by reference in their entireties.

In some embodiments, the bioluminescent reporter protein comprises NANOLUC (See U.S. Pat. App. Nos. 2010/0281552 and 2012/0174242, herein incorporated by reference in their entireties). In some embodiments, the bioluminescent reporter protein comprises a polypeptide with at least 70% identity (e.g., >70%, >80%, >90%, >95%) to SEQ ID NO: 1 that retains bioluminescent characteristics. In certain embodiments, the use of the NANOLUC enzyme, or a variant thereof, provides features (e.g., signal intensity, brightness, high light output, narrow spectrum, etc.) that enable the use of the BRET assays described herein. In some embodiments, the high light output of NANOLUC enables the low concentration (e.g., <1 μM, <100 nM, <10 nm, <1 nm, etc.) of assay components, e.g., DNA encoding NANOLUC, useful to carry out assays under physiologically relevant conditions. In some embodiments, NANOLUC enables the use of BRET in characterizing cellular targets identified in a phenotypic screen. In some embodiments, BRET efficiency is significantly enhanced by the technical features of NANOLUC as an energy donor compared to other luciferases. For example, NANOLUC is significantly brighter than other luciferases commonly used for BRET, thereby allowing energy transfer to be quantitated at lower expression levels which are more suitable for maintaining relevant biology within a cell. In some embodiments, the narrow emission spectrum of NANOLUC increases the dynamic range by reducing spectral cross-over in the acceptor channel. In some embodiments, the dynamic range can be further increased by using long-wavelength acceptors that emit in the near-red region of the spectrum (600 to 650 nm). In some embodiments, evaluation of multiple dye ligands during development of embodiments of the present invention revealed that rhodamine analogs (e.g., carboxy rhodamine analog), such as those described in U.S. patent application Ser. No. 13/682,589, herein incorporated by reference in its entirety, delivers an optimal dynamic range for use with NANOLUC and/or in the BRET applications described herein.

In some embodiments, the fusion protein comprises a DNA polymerase theta (PolΘ). A DNA polymerase theta refers to an enzyme encoded by the POLQ gene. In some embodiments, the DNA polymerase theta refers to human DNA polymerase theta encoded by the human POLQ gene (NCBI Gene ID 10721). In some embodiments, DNA polymerase theta refers to a polypeptide comprising an amino acid sequence having at least 80% sequence identity (e.g. at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identity to)

(SEQ ID NO: 2)
MNLLRRSGKRRRSESGSDSFSGSGGDSSASPQFLSGSVLSPPPGLGRCLK
AAAAGECKPTVPDYERDKLLLANWGLPKAVLEKYHSFGVKKMFEWQAECL
LLGQVLEGKNLVYSAPTSAGKTLVAELLILKRVLEMRKKALFILPFVSVA
KEKKYYLQSLFQEVGIKVDGYMGSTSPSRHFSSLDIAVCTIERANGLINR
LIEENKMDLLGMVVVDELHMLGDSHRGYLLELLLTKICYITRKSASCQAD
LASSLSNAVQIVGMSATLPNLELVASWLNAELYHTDFRPVPLLESVKVGN
SIYDSSMKLVREFEPMLQVKGDEDHVVSLCYETICDNHSVLLFCPSKKWC
EKLADIIAREFYNLHHQAEGLVKPSECPPVILEQKELLEVMDQLRRLPSG
LDSVLQKTVPWGVAFHHAGLTFEERDIIEGAFRQGLIRVLAATSTLSSGV
NLPARRVIIRTPIFGGRPLDILTYKQMVGRAGRKGVDTVGESILICKNSE
KSKGIALLQGSLKPVRSCLQRREGEEVTGSMIRAILEIIVGGVASTSQDM
HTYAACTFLAASMKEGKQGIQRNQESVQLGAIEACVMWLLENEFIQSTEA
SDGTEGKVYHPTHLGSATLSSSLSPADTLDIFADLQRAMKGFVLENDLHI
LYLVTPMFEDWTTIDWYRFFCLWEKLPTSMKRVAELVGVEEGFLARCVKG
KVVARTERQHRQMAIHKRFFTSLVLLDLISEVPLREINQKYGCNRGQIQS
LQQSAAVYAGMITVFSNRLGWHNMELLLSQFQKRLTFGIQRELCDLVRVS
LLNAQRARVLYASGFHTVADLARANIVEVEVILKNAVPFKSARKAVDEEE
EAVEERRNMRTIWVTGRKGLTEREAAALIVEEARMILQQDLVEMGVQWNP
CALLHSSTCSLTHSESEVKEHTFISQTKSSYKKLTSKNKSNTIFSDSYIK
HSPNIVQDLNKSREHTSSFNCNFQNGNQEHQTCSIFRARKRASLDINKEK
PGASQNEGKTSDKKVVQTFSQKTKKAPLNFNSEKMSRSFRSWKRRKHLKR
SRDSSPLKDSGACRIHLQGQTLSNPSLCEDPFTLDEKKTEFRNSGPFAKN
VSLSGKEKDNKTSFPLQIKQNCSWNITLTNDNFVEHIVTGSQSKNVTCQA
TSVVSEKGRGVAVEAEKINEVLIQNGSKNQNVYMKHHDIHPINQYLRKQS
HEQTSTITKQKNIIERQMPCEAVSSYINRDSNVTINCERIKLNTEENKPS
HFQALGDDISRTVIPSEVLPSAGAFSKSEGQHENFLNISRLQEKTGTYTT
NKTKNNHVSDLGLVLCDFEDSFYLDTQSEKIIQQMATENAKLGAKDTNLA
AGIMQKSLVQQNSMNSFQKECHIPFPAEQHPLGATKIDHLDLKTVGTMKQ
SSDSHGVDILTPESPIFHSPILLEENGLFLKKNEVSVTDSQLNSFLQGYQ
TQETVKPVILLIPQKRTPTGVEGECLPVPETSLNMSDSLLFDSFSDDYLV
KEQLPDMQMKEPLPSEVTSNHFSDSLCLQEDLIKKSNVNENQDTHQQLTC
SNDESIIFSEMDSVQMVEALDNVDIFPVQEKNHTVVSPRALELSDPVLDE
HHQGDQDGGDQDERAEKSKLTGTRQNHSFIWSGASFDLSPGLQRILDKVS
SPLENEKLKSMTINFSSLNRKNTELNEEQEVISNLETKQVQGISFSSNNE
VKSKIEMLENNANHDETSSLLPRKESNIVDDNGLIPPTPIPTSASKLTFP
GILETPVNPWKTNNVLQPGESYLFGSPSDIKNHDLSPGSRNGFKDNSPIS
DTSFSLQLSQDGLQLTPASSSSESLSIIDVASDQNLFQTFIKEWRCKKRF
SISLACEKIRSLTSSKTATIGSRFKQASSPQEIPIRDDGFPIKGCDDTLV
VGLAVCWGGRDAYYFSLQKEQKHSEISASLVPPSLDPSLTLKDRMWYLQS
CLRKESDKECSVVIYDFIQSYKILLLSCGISLEQSYEDPKVACWLLDPDS
QEPTLHSIVTSFLPHELPLLEGMETSQGIQSLGLNAGSEHSGRYRASVES
ILIFNSMNQLNSLLQKENLQDVFRKVEMPSQYCLALLELNGIGFSTAECE
SQKHIMQAKLDAIETQAYQLAGHSFSFTSSDDIAEVLFLELKLPPNREMK
NQGSKKTLGSTRRGIDNGRKLRLGRQFSTSKDVLNKLKALHPLPGLILEW
RRITNAITKVVFPLQREKCLNPFLGMERIYPVSQSHTATGRITFTEPNIQ
NVPRDFEIKMPTLVGESPPSQAVGKGLLPMGRGKYKKGFSVNPRCQAQME
ERAADRGMPFSISMRHAFVPFPGGSILAADYSQLELRILAHLSHDRRLIQ
VLNTGADVFRSIAAEWKMIEPESVGDDLRQQAKQICYGIIYGMGAKSLGE
QMGIKENDAACYIDSFKSRYTGINQFMTETVKNCKRDGFVQTILGRRRYL
PGIKDNNPYRKAHAERQAINTIVQGSAADIVKIATVNIQKQLETFHSTFK
SHGHREGMLQSDQTGLSRKRKLQGMFCPIRGGFFILQLHDELLYEVAEED
VVQVAQIVKNEMESAVKLSVKLKVKVKIGASWGELKDFDV.

In some embodiments, a DNA polymerase theta refers to a polypeptide having one or more mutations relative to SEQ ID NO: 2 (e.g., truncations, deletions, insertions, substitutions, etc.), that still retains polymerase activity.

In some embodiments, the bioluminescent reporter protein (e.g. luciferase) and the DNA polymerase theta are fused, tethered, connected, etc. by any suitable structure or mechanism (e.g., expressed as a fusion construct (e.g., with or without peptide linker), chemically linked (e.g., through covalent or non-covalent bonds), enzymatically linked, linked by a linker (e.g., peptide, nucleic acid, other polymer (e.g., ester linkage, PEG linker, carbon chain, etc.)). In some embodiments, an amino acid chain (e.g., 3-100 amino acids) is used to connect the DNA polymerase theta and the bioluminescent reporter protein (e.g., luciferase). In some embodiments, the structure and/or function of each of the DNA polymerase theta and the bioluminescent reporter protein are not significantly impacted by fusion or the presence of the linker. In certain embodiments, a linker allows fusion without loss of activity or one or both of the elements. In other embodiments, an amino acid linker properly spaces and/or orients the bioluminescent reporter protein for energy transfer with the fluorophore.

### DNA Fragments

In some aspects, provided herein are DNA fragments. In some embodiments, provided herein are DNA fragments that bind coordinatively to DNA polymerase theta (PolΘ) with another entity. For example, in some embodiments provided herein are DNA fragments that bind coordinatively to DNA polymerase theta with a tracer (e.g., a compound of formula Ia, a compound of formula Ib). As another example, in some embodiments provided herein are DNA fragments that bind coordinatively to DNA polymerase theta with an inhibitor of DNA polymerase theta. As used herein, the terms “bind coordinatively” or “coordinative binding” refers to an uncompetitive form of binding wherein drug binding is potentiated by the presence of the protein substrate (in this case, DNA fragments). In some embodiments, binding of one ligand (e.g., DNA fragments) enhances the ability of the other ligand (e.g., a tracer) to bind to the target (e.g., DNA polymerase theta).

In some embodiments, provided herein are DNA fragments that bind to the polymerase domain of DNA polymerase. In some embodiments, the DNA fragments bind coordinatively to the polymerase domain of DNA polymerase with a compound of formula Ia or a compound of formula Ib. In some embodiments, the DNA fragments bind coordinatively to the polymerase domain of DNA polymerase present in a fusion protein provided herein. In some embodiments, coordinative binding of the DNA fragments and the tracer (e.g. the compound of formula Ia, the compound of formula Ib) to the polymerase domain of DNA polymerase present in a fusion protein provided herein facilitates transfer of energy from the bioluminescent reporter protein (e.g., luciferase) to the fluorophore present within the tracer, such as by BRET. Accordingly, coordinative binding of the DNA fragments and the tracer (e.g. compound of formula Ia, compound of formula Ib) to the polymerase domain of DNA polymerase present in the fusion protein facilitates evaluation of target engagement by a detectable BRET signal. In some embodiments, addition of an agent that engages with (e.g., binds to, inhibits) DNA polymerase theta causes displacement of the tracer (e.g., compound of formula I) and a detectable reduction in BRET signal.

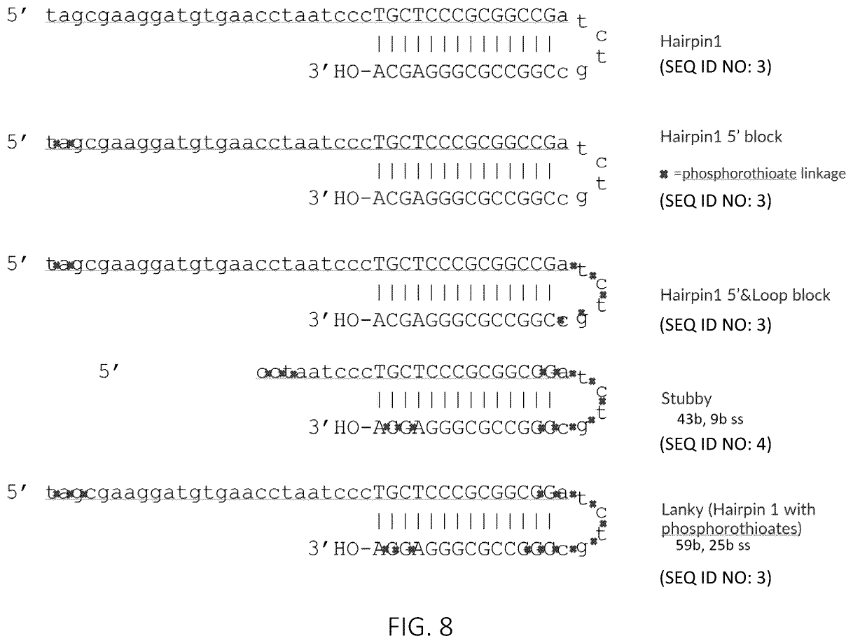

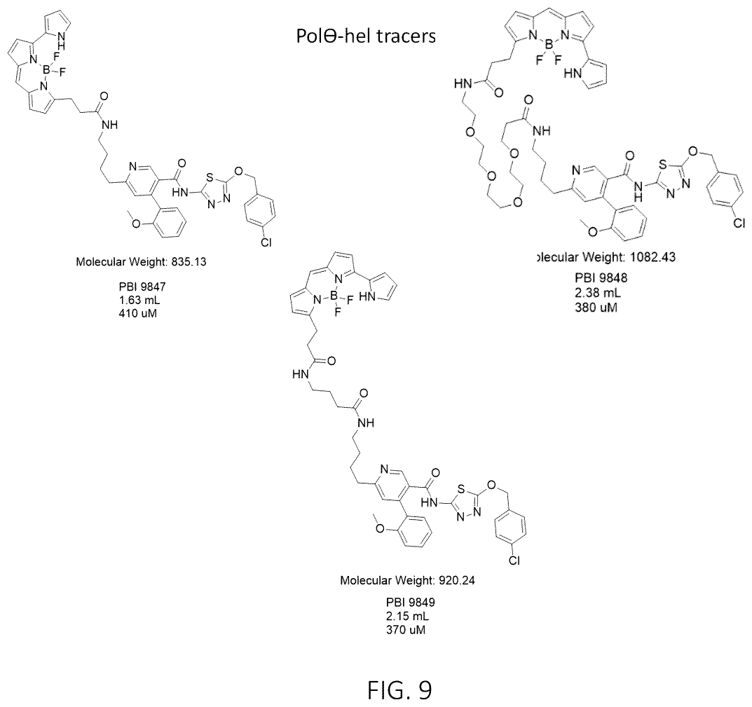

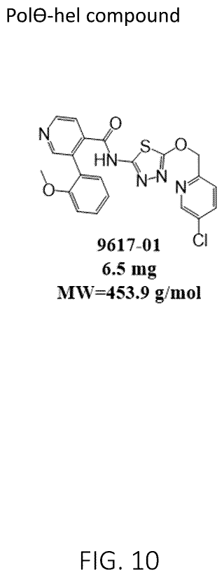

Any suitable size, sequence, and structure of DNA fragments may be used. In some embodiments, a DNA fragment comprises two annealed single stranded DNAs. In some embodiments, a DNA fragment forms a hairpin structure with a single-stranded overhang. In some embodiments, the hairpin structure comprises a loop, a double-stranded stem, and a single-stranded overhang. Exemplary hairpin DNA structures are shown in FIG. 8. However, the application is not limited to hairpin DNA structures or to the exemplary hairpin DNA structures shown therein. In some embodiments, the DNA fragment comprises 10-200 bases. In some embodiments, the loop comprises 3-50 bases. In some embodiments, the double-stranded stem comprises 4-100 bases. In some embodiments, the single-stranded overhang comprises 2-50 bases. In some embodiments, the single-stranded overhang comprises 5-30 bases. In some embodiments, the single-stranded overhang comprises 10 or fewer bases. A DNA fragment comprising a single-stranded overhang with 10 or fewer bases is referred to herein as a “stubby” DNA hairpin. In some embodiments, the DNA fragment comprises a single-stranded overhang with more than 10 bases. In some embodiments, the DNA fragment comprises a single-stranded overhang with 20-30 bases. A DNA fragment comprising a single-stranded overhang with more than 10 bases (e.g., 20-30 bases) is referred to herein as a “lanky” DNA fragment.

### Systems and Assays for Target Engagement

In some aspects, provided herein are systems. In some embodiments, provided herein are systems for use in an assay for target engagement with DNA polymerase theta (PolΘ). In some embodiments, the assay is a BRET assay. Accordingly, in some embodiments, the systems are referred to herein as BRET assay systems. Exemplary BRET assays are described in U.S. Pat. Nos. 10,067,149 and 10,024,862, the entire contents of each of which are incorporated herein by reference for all purposes.

In some embodiments, provided herein is a system (e.g., a BRET assay system) comprising a compound of formula (I), an energy transfer acceptor, a fusion protein comprising a bioluminescent reporter and a DNA polymerase theta, and a DNA fragment, each of which are described in detail in the sections above. In some embodiments, the compound of formula (I) is conjugated to the energy transfer acceptor, thereby creating a tracer. In some embodiments, the energy transfer acceptor is a chromophore. In some embodiments, the energy transfer acceptor is a fluorophore. In some embodiments, a tracer (e.g., fluorescent tracer) comprising a compound of formula (I) conjugated to an energy transfer acceptor interacts with (e.g., binds to) the polymerase domain of DNA polymerase theta. Accordingly, in some embodiments the tracer comprising a compound of formula (I) conjugated to an energy transfer acceptor binds to the polymerase domain of the DNA polymerase theta present within the fusion protein comprising the DNA polymerase theta and a bioluminescent reporter, as described above. In some embodiments, the tracer binds coordinatively with the one or more DNA fragments to the polymerase domain of DNA polymerase theta. In some embodiments, the system further comprises a substrate for the bioluminescent reporter. In some embodiments, upon coordinative binding of the tracer and the one or more DNA fragments to the polymerase domain of DNA polymerase theta, conversion of the substrate to a reaction product by the bioluminescent reporter results in BRET and increased fluorescence emission from the energy transfer acceptor (e.g. fluorophore). In some embodiments, coordinative binding of the one or more DNA fragments and the tracer to the DNA polymerase theta (e.g., within the fusion protein) brings the bioluminescent reporter and the energy acceptor into proximity to one another, thus facilitating the BRET reaction. Accordingly, the system can evaluate interaction between the fusion protein and the tracer. Moreover, the system can evaluate whether an agent engages DNA polymerase theta. Specifically, an agent that engages (e.g., binds) to the polymerase domain of DNA polymerase theta displaces the fluorescent tracer from DNA polymerase theta, thereby reducing the BRET signal by disrupting energy transfer from the bioluminescent reporter protein to the energy acceptor (e.g., fluorophore) of the tracer.

In some embodiments, provided herein is a system comprising a compound of formula (II), an energy transfer acceptor, and a fusion protein. In some embodiments, the energy transfer acceptor is a fluorophore. In some embodiments, a tracer (e.g., fluorescent tracer) comprising a compound of formula (II) conjugated to an energy acceptor interacts with (e.g., binds to) the helicase domain of DNA polymerase theta, including the helicase domain of a DNA polymerase theta present within a fusion protein comprising a DNA polymerase theta and a bioluminescent reporter, as described above. In some embodiments, the system further comprises a substrate for the bioluminescent reporter. In some embodiments, upon binding of the tracer to the helicase domain of DNA polymerase theta, conversion of the substrate to a reaction product by the bioluminescent reporter results in BRET and increased fluorescence emission from the energy acceptor (e.g. fluorophore). In some embodiments, binding of the tracer to the DNA polymerase theta (e.g., within the fusion protein) brings the bioluminescent reporter and the energy acceptor into proximity to one another, thus facilitating the BRET reaction. Accordingly, the system can evaluate interaction between the fusion protein and the tracer. Moreover, the system can evaluate whether an agent engages DNA polymerase theta. Specifically, an agent that engages (e.g., binds) to the helicase domain of DNA polymerase theta displaces the fluorescent tracer from DNA polymerase theta, thereby reducing the BRET signal by disrupting energy transfer from the bioluminescent reporter protein to the energy acceptor (e.g., fluorophore) of the tracer.

In some embodiments, a substrate for the bioluminescent reporter protein is provided. In some embodiments, the bioluminescent reporter protein converts the substrate into a reaction product and releases light energy as a by-product. In some embodiments, the substrate is a substrate for a luciferase enzyme. In some embodiments, the substrate is a structural variant or derivative of coelenterazine (e.g., furimazine). In some embodiments, the substrate is a substrate for a modified, enhanced luciferase enzyme from Oplophorus, e.g., NANOLUC enzyme from Promega Corporation (e.g., SEQ ID NO: 1). In some embodiments, a pro-substrate for the bioluminescent reporter protein is provided, which produces a substrate through a chemical or physical process (e.g., hydrolysis, enzymatic reaction, photo-cleavage, etc.). In some embodiments, the pro-substrate comprises coelenterazine, a coelenterazine derivative, a structural or functional equivalent of coelenterazine, a molecule substantially equivalent to coelenterazine (e.g., structurally and/or functionally), or molecule functionally or structurally similar to coelenterazine. In some embodiments, the bioluminescent reporter protein converts the coelenterazine, coelenterazine derivative, structural or functional equivalent of coelenterazine, or substantial equivalent to coelenterazine into coelenteramide, a coelenteramide derivative, a structural or functional equivalent of coelenteramide, or a substantial equivalent to coelenteramide and releases light energy as a by-product.

In some embodiments, provided herein are assays for target engagement with DNA polymerase theta. In some embodiments, the assays are performed using the systems or the individual components thereof described herein. In some embodiments, interaction of a moiety with DNA polymerase theta is detected, characterized, quantified, analyzed, etc. through the detection/measurement of a signal produced by signal transfer (e.g., transfer of energy (e.g., fluorescence, light energy, resonance, by BRET, etc.)) between the bioluminescent reporter protein within the fusion protein (e.g. connected, fused, tethered, linked, etc. to the DNA polymerase theta) and the energy acceptor of the tracer. For example, in some embodiments the bioluminescent reporter protein emits energy upon interaction with its substrate, and that emitted energy is absorbed by energy acceptor (e.g., fluorophore) under high affinity conditions. As used herein, the term “high affinity” or “high affinity conditions” describes a condition wherein the tracer (e.g., compound of formula I or formula II bound to an energy acceptor) binds to DNA polymerase theta (e.g., within a fusion protein comprising the DNA polymerase theta and a bioluminescent reporter protein) with a stronger affinity than polymerase theta alone, thus bringing the bioluminescent reporter protein in sufficient proximity to the energy acceptor such that energy transfer can occur. In contrast, “low affinity” or “low affinity conditions” refers to a condition wherein the tracer is not sufficiently bound to or is displaced from the polymerase theta of the fusion protein, such that the bioluminescent reporter of the fusion protein is not sufficiently close to the energy acceptor of the tracer to facilitate energy transfer. Accordingly, in some embodiments under “high affinity” conditions a high BRET signal occurs, whereas under “low affinity” conditions a low BRET signal occurs. In some embodiments, high affinity conditions require one or more DNA fragments to be present to bind coordinatively with the tracer to the DNA polymerase theta. For example, in some embodiments tracers comprising a compound of formula (I) bind coordinatively with one or more DNA fragments to DNA polymerase theta to achieve a high-affinity state, whereas in the absence of the one or more DNA fragments a high-affinity state cannot be achieved (e.g., a low-affinity condition exists). In some embodiments, high affinity conditions can be achieved using a tracer comprising a compound of formula (II) without the requirement for added DNA fragments.

In some embodiments, the energy acceptor absorbs energy from the bioluminescent reporter under high affinity conditions and emits energy at a measurable different energy from the energy emitted by the bioluminescent reporter. Accordingly, the energy emitted from the energy acceptor (e.g., within the tracer) can be detected/measured/quantified separately from the energy emitted by the bioluminescent reporter. In some embodiments, the duration, kinetics, affinity, strength, and/or specificity of the binding of an agent to DNA polymerase theta is detected, measured, quantified, determined, interrogated, etc., based on measurement of the signal output of the energy acceptor under various conditions. In some embodiments, the energy acceptor (e.g., fluorophore) and the bioluminescent reporter are selected that exhibit sufficient overlap of emission (e.g., of bioluminescent reporter) and excitation (e.g., of fluorophore) spectra to provide efficient energy transfer between the two (e.g., by non-radiative dipole-dipole coupling). In some embodiments, the peak emission of the bioluminescent reporter is substantially separated from the peak emission of the fluorophore, for example by at least 80 nm, 100 nm, 120 nm, 140 nm, etc., in wavelength. In particular embodiments, the Forster distance of the fluorophore and bioluminescent reporter pair is small (e.g., <20 nm, <10 nm, <5 nm, <3 nm, etc.). In such embodiments, the short Forster distance results in the requirement that the fluorophore and bioluminescent reporter must be brought into very close proximity for energy transfer to occur. Therefore, the short Forster distance reduces aberrant and/or background signal (e.g., created by diffusing fluorophore and/or reporter).

In certain embodiments, a fluorophore and bioluminescent reporter pair are selected that are sufficiently bright to allow detection of the transferred signal at a native abundance (or near native abundance) of the protein of interest and/or the cellular target fused to the bioluminescent reporter. In some embodiments, should either the selected fluorophore or bioluminescent reporter produce insufficient energy (light) emission, either the fusion of the cellular target and bioluminescent reporter will need to be overexpressed (e.g., beyond native abundance, beyond a biologically relevant level, etc.), and/or the amount of fluorophore-tethered bioactive agent will have to be increased (e.g., to a potentially toxic level, beyond a physiologically relevant level, above the amount when kinetic experiments can be performed, etc.). In some embodiments, sufficient brightness of the bioluminescent reporter and fluorophore allows detection of bioactive agent and cellular target interaction at a suitable range of concentrations and ratios.

In some embodiments, the fusion protein comprising the bioluminescent reporter and a DNA polymerase theta is expressed in cells in which an assay is to be performed. In some embodiments, the fusion protein is expressed at a natural cellular abundance (e.g., relative to the expression of native DNA polymerase theta or at a level appropriate for proper biological function of DNA polymerase theta). In some embodiments, interaction of an agent with DNA polymerase theta is detected intracellularly. For example, in some embodiments cells are transfected with an expression vector (e.g., plasmid) encoding the fusion protein. In some embodiments, the tracer is added extracellularly (e.g., added to the culture medium) and enters the cell via diffusion, active transport, passive transport, endocytosis, or any suitable mechanism. In some embodiments, varying amount of tracer is added to the cells to assay binding kinetics, assay binding affinity, provide sufficient signal, etc. In some embodiments, the tracer is cell permeable. In some embodiments, the tracer is modified to contain a cell-penetrating moiety. In some embodiments, the cell is permeabilized to permit entry of the tracer into the cell. In some embodiments, the cell is electroporated to permit entry of the tracer into the cell. A substrate for the bioluminescent reporter protein is added to the cell prior, concurrently or subsequent to the addition of the tracer. Interaction between the tracer and the fusion protein (e.g., binding of the tracer to the DNA polymerase theta) results in BRET signal, indicative of energy transfer from the bioluminescent reporter of the fusion protein to the energy acceptor of the tracer. Accordingly, detection of a fluorescent signal from the fluorophore (as the result of BRET) indicates a high affinity state between the tracer and the fusion protein (e.g., binding between the tracer and the polymerase domain of DNA polymerase theta, binding between the tracer and the helicase domain of DNA polymerase theta). In some embodiments, such as embodiments wherein the tracer comprises a compound of formula (I), one or more DNA fragments are introduced into the cell. In other embodiments, one or more DNA fragments are not necessary to achieve a high affinity state between the tracer and the fusion protein. The one or more DNA fragments may be introduced into the cell prior to, concurrently with, or following addition of the tracer, and may be introduced into the cell by any suitable means (e.g., permeabilization, electroporation, etc.).

In some embodiments, characteristics of the interaction between the tracer and DNA polymerase theta are interrogated by altering the cellular conditions or the conditions of the system. For example, in some embodiments, an agent can be added to the cell to determine whether the agent competes with/displaces the tracer, thereby reducing BRET signal. For example, in some embodiments a putative competitive binder of DNA polymerase theta is added to the cell to compete with the tracer. In some embodiments, an agent can be added to the system, and the effect of the agent on the BRET signal can be evaluated. Agents that reduce BRET signal are determined to be agents that engage with DNA polymerase theta (e.g., bind to, inhibit, etc.) In contrast, agents that do not reduce BRET signal do not disrupt the binding between the tracer and the DNA polymerase theta. Accordingly, the systems and methods provided herein find use in methods of determining whether an agent is an engager of DNA polymerase theta, such as for drug discovery.

Some embodiments described herein find use in drug discovery, drug validation, drug target discovery, or drug target validation. In some embodiments, the relative binding affinity of an agent for DNA polymerase theta (e.g., in solution, in a lysate, on a surface, in a cell, etc.) can be determined by the ability of the agent to displace a tracer that has been tethered to a fluorophore. Specifically, higher binding affinity of an agent relative to the tracer is indicated by requiring a lower concentration of the agent required to displace the tracer. Displacement of the tracer is determined by the loss or reduction of energy transfer from the bioluminescent reporter protein fused to the DNA polymerase theta. In some embodiments, the concentration of the agent needed to displace the tracer is used to estimate the binding EC50 or the inhibition constant (Ki) for the agent.

In some embodiments, a collection of compounds which may have unknown binding affinity to DNA polymerase theta be screened for their ability to bind to DNA polymerase theta fused to a bioluminescent protein by determining their ability to displace the tracer from the DNA polymerase theta. In some embodiments, such as when compounds of formula (I) are used in the tracer, such experiments are conducted in the presence of one or more DNA fragments. In other embodiments, such as when compounds of formula (II) are used in the tracer, the one or more DNA fragments may be used but are not necessary. In some embodiments, compounds that displace a tracer comprising a compound of formula (I) are identified as engagers of the polymerase domain of DNA polymerase theta. In some embodiments, compounds that displace a tracer comprising a compound of formula (II) are identified as engagers of the helicase domain of DNA polymerase theta.

### EXPERIMENTAL

### Example 1

### Target Engagement at Polymerase Domain of Polymerase Theta in Permeabilized Cells

During development of the target engagement assay for Polymerase Theta (POL Theta; PolΘ) described herein, experiments were conducted to evaluate live cell target engagement via addition of DNA fragments. 4,000,000 HEK293 cells were transfected with POLQ/NanoLuc (Nluc) fusions expressed from pFN32K plasmids. Transfections were performed using 3:1 FuGENE HD:plasmid ratios. 1 uM (final) of each plasmid was co-transfected with Transfection Carrier DNA/pGEM as a decoy DNA. 20-24 hours post transfection, cells were treated with 1 uM (final) DNA fragment. Cells were then treated for 2 hours in the presence of 50 μg/mL digitonin and 1 uM (final) POLQ tracers 9623, 9624, 9625, 9685, 9686, and 9687, and 10 uM (final) control inhibitor, 9618. After incubation, NanoBRET-TE substrate solution was added to a final concentration of 1×, and BRET was measured on a Glomax Discover plate reader.

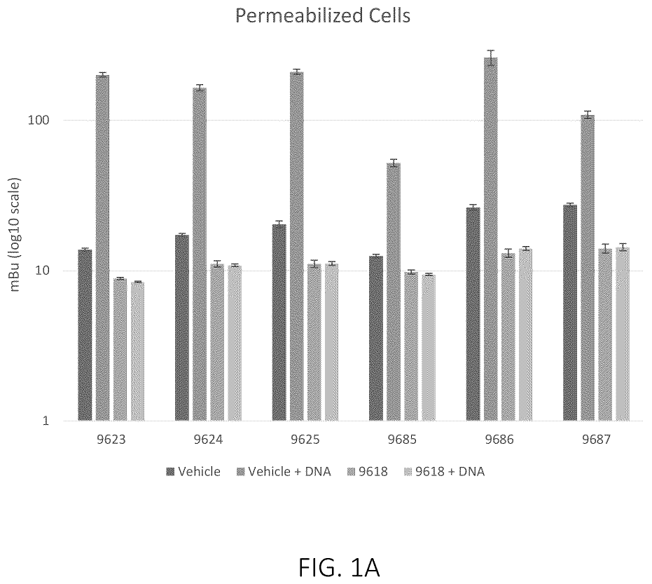

As seen in FIG. 1A, in each case, addition of DNA fragments potentiated the NanoBRET signal with the tracer. Enhanced target engagement of unmodified drug was also observed in the presence of DNA fragment as seen in FIG. 1B. Specifically, as shown in FIG. 1B, bottom left panel, BRET signal is reduced with increasing concentrations of the polymerase theta inhibitor 9618 when DNA fragments are present, indicating high affinity of the tracer with polymerase theta and enhanced target engagement of the inhibitor with polymerase theta when coordinative binding of DNA fragments occurs. In contrast, as shown in the bottom left panel, a low BRET signal was seen in the absence of DNA fragments, and the low signal did not significantly decrease upon addition of increasing concentrations of polymerase theta inhibitor, indicating low affinity between polymerase theta, the tracer, and the inhibitor in the absence of DNA fragments.

### Example 2

### Enhancement of Target Engagement at Pol Domain of POL Theta in Live Cells

During development of the target engagement assay for Polymerase Theta (POL Theta; PolΘ) described herein, experiments were conducted to evaluate live cell target engagement via ectopic delivery of DNA fragments. 4,000,000 HEK293 cells were transfected with POLQ-NanoLuc (Nluc) fusions expressed from pFN32K plasmids. Transfections were performed using 3:1 FuGENE HD:plasmid ratios. 1 ug of the POLQ-NLuc plasmid was co-transfected with 9 ug Transfection Carrier DNA/pGEM as a decoy DNA. 40-48 hours post transfection, cells were resuspended and electroporated using a BioRad or Mirus electroporator with 4.9-14.3 uM DNA fragments as indicated in the figures. After electroporation, cells were optionally pelleted and resuspended in OptiMEM, and seeded into 96-well plates at 20,000 cells per well and treated for 2 hours in the presence of 1 uM POLQ tracer 9686 and 10 uM control inhibitor 9618. After incubation, NanoBRET-TE substrate/inhibitor solution was added to a final concentration of 1×, and BRET was measured on a Glomax Discover plate reader.

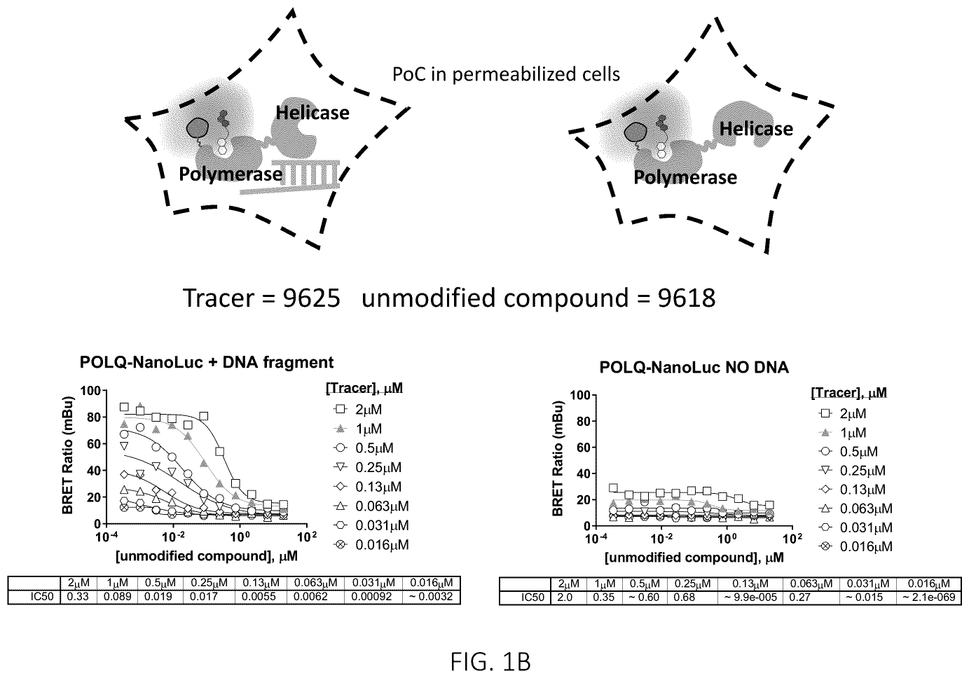

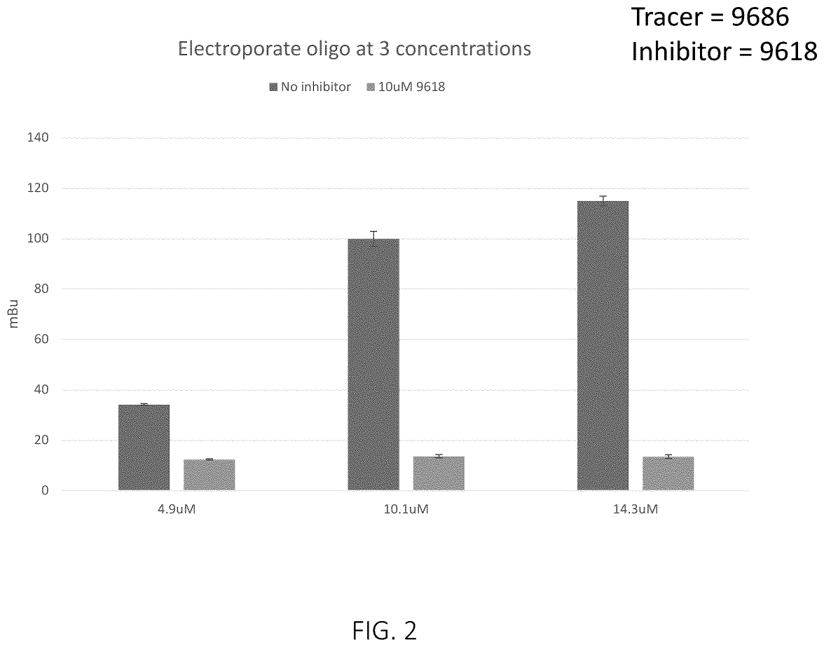

As seen in FIG. 2, in each case, addition of DNA fragments potentiated the NanoBRET signal with the tracer. Enhanced target engagement of unmodified drug was also observed in the presence of DNA fragment.

### Example 3

### Live Cell Target Engagement with Full Length Polymerase Theta with Pol Domain Tracer

During development of the target engagement assay for Polymerase Theta (POL Theta; PolΘ) described herein, experiments were conducted to evaluate live cell target engagement via ectopic delivery of DNA fragments. 4,000,000 HEK293 cells were transfected with POLQ-NanoLuc (Nluc) fusions expressed from pFN32K plasmids. Transfections were performed using 3:1 FuGENE HD:plasmid ratios. 1 ug of the POLQ-NLuc plasmid was co-transfected with 9 ug Transfection Carrier DNA/pGEM as a decoy DNA. 20-24 hours post transfection, cells were resuspended and electroporated using a Mirus electroporator with 10.7 uM DNA fragments. After electroporation, cells were resuspended in OptiMEM, and seeded into 96-well plates at 20,000 cells per well and treated for 2 hours in the presence of various concentrations of POLQ tracer 9686 and control inhibitor 9618. After incubation, NanoBRET-TE substrate/inhibitor solution was added to a final concentration of 1×, and BRET was measured on a Glomax Discover plate reader.

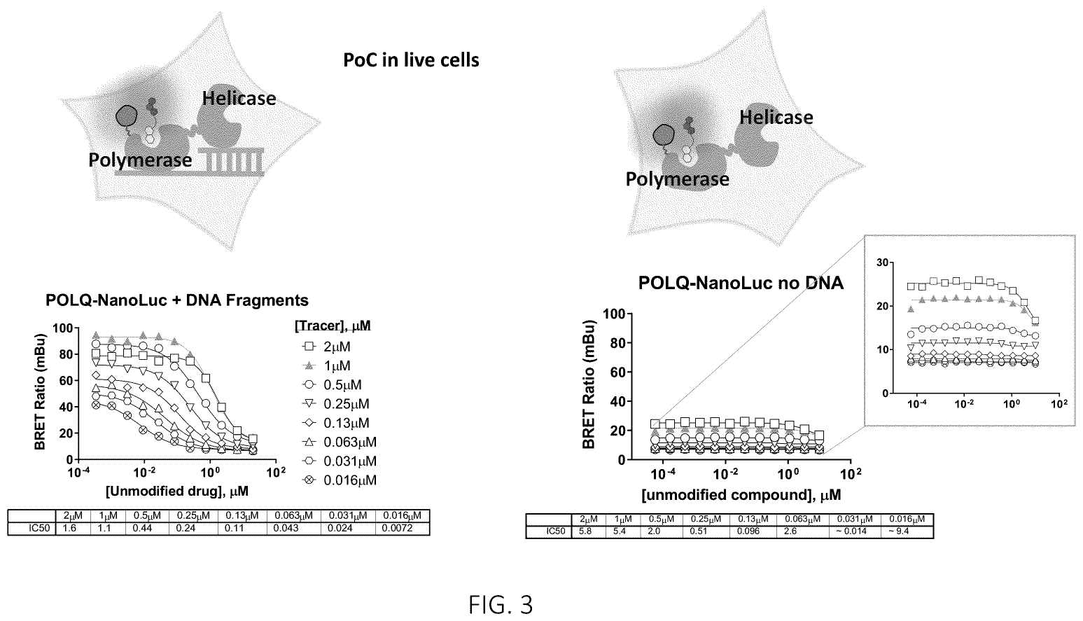

As seen in FIG. 3, addition of DNA fragments potentiated the NanoBRET signal with the tracer and a high affinity state was observed.

### Example 4

### Live Cell Target Engagement with Various Configurations of DNA Fragments

During development of the target engagement assay for Polymerase Theta (POL Theta; PolΘ) described herein, experiments were conducted to evaluate live cell target engagement various configurations of DNA fragments, e.g., “stubby” or “lanky.” The 59 base Lanky 1 (T*A*G*CGAAGGATGTGAACCTAATCCCTGCTCCCGCGGCC*G*A*T*C*T*G*C*C*G*GCCGCGGGA*G*C*A) and 43 base Stubby (C*C*T*AATCCCTGCTCCCGCGGCC*G*A*T*C*T*G*C*C*G*GCCGCGGGA*G*C*A) oligonucleotides both form a hairpin with a 6 base loop with a 14 base pair double-stranded region. Lanky 2 has 25 base single-stranded 5′overhang, whereas Stubby has a 9 base single-stranded 5′overhang.

In a first experiment, 4,000,000 HEK293 cells were transfected with POLQ-NanoLuc (Nluc) fusions expressed from pFN32K plasmids. Transfections were performed using 3:1 FuGENE HD:plasmid ratios. 1 ug of the POLQ-NLuc plasmid was co-transfected with 9 ug Transfection Carrier DNA/pGEM as a decoy DNA. 20-24 hours post transfection, cells were resuspended and electroporated using a BioRad electroporator with 10.7 uM DNA fragments Lanky 1, Lanky2, and Stubby. After electroporation, cells were resuspended in OptiMEM, and seeded into 96-well plates at 20,000 cells per well and treated for 2 hours in the presence of 1 uM POLQ tracer 9686 and 10 uM control inhibitor 9618. After incubation, NanoBRET-TE substrate/inhibitor solution was added to a final concentration of 1×, and BRET was measured on a Glomax Discover plate reader.

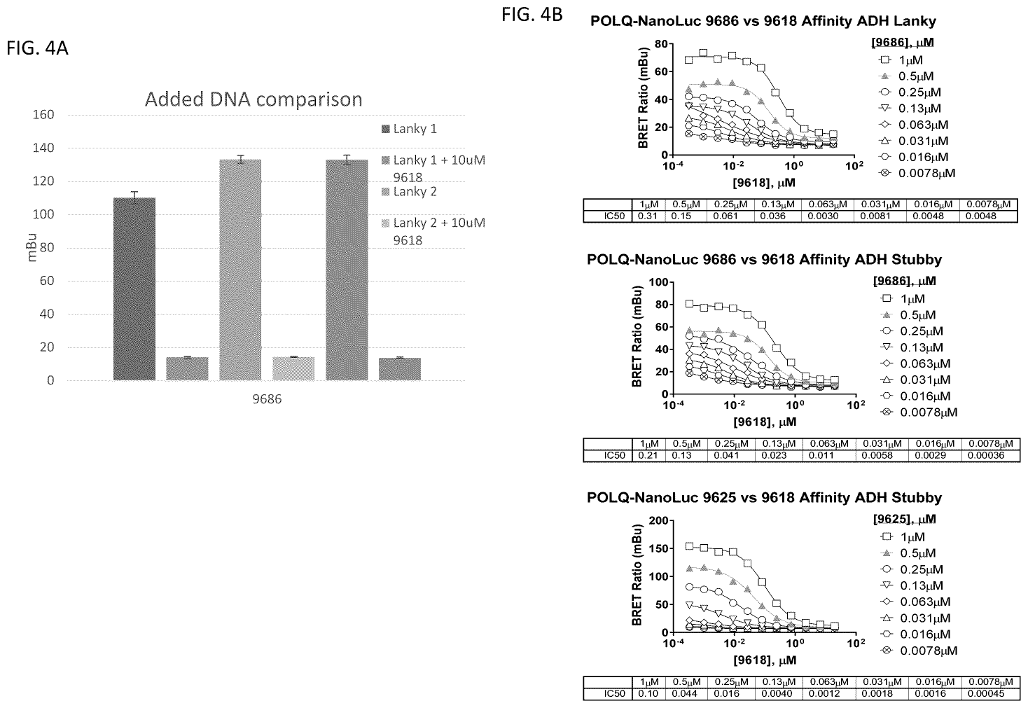

As seen in FIG. 4A, Lanky DNA fragments behave similarly. Lanky 1 and Lanky 2 are identical with respect to oligonucleotide and electroporation conditions except Lanky 2 sample cells were pelleted and resuspended in OptiMEM as a control to show that extracellular DNA does not contribute to the observed NanoBRET.

In another experiment, 4,000,000 HEK293 cells were transfected with POLQ-NLuc fusions expressed from pFN32K plasmids. Transfections were performed using 3:1 FuGENE HD:plasmid ratios. 2 ug of the POLQ-NLuc plasmid was co-transfected with 8 ug Transfection Carrier DNA/pGEM as a decoy DNA. 20-24 hours post transfection, cells were resuspended and electroporated using a BioRad electroporator with 4.6 uM DNA fragments Lanky or Stubby. After electroporation, cells were passed through a cell strainer and resuspended in OptiMEM, seeded into 96-well plates at 20,000 cells per well, and treated for 2 hours in the presence of various concentrations of POLQ tracer 9686 or 9625 and various concentrations of control inhibitor 9618. After incubation, NanoBRET-TE substrate/inhibitor solution was added to a final concentration of 1×, and BRET was measured on a Glomax Discover plate reader.

As seen in FIG. 4B, Stubby and Lanky DNA fragments behave similarly with the Lanky DNA fragment, and the Stubby DNA fragment works well with either POLQ tracer.

### Example 5

### Target Engagement at POL Theta Helicase Domain in Live Cells

During development of the target engagement assay for Polymerase Theta (POL Theta; PolΘ) described herein, experiments were conducted to evaluate live cell target engagement via ectopic delivery of DNA fragments. 4,000,000 HEK293 cells were transfected with NanoLuc (Nluc)-POLQ fusions expressed from pFN31K plasmids. Transfections were performed using 3:1 FuGENE HD:plasmid ratios. Transfections were performed using 3:1 FuGENE HD:plasmid ratios. 1 ug of the NLuc-POLQ plasmid was co-transfected with 9 ug Transfection Carrier DNA/pGEM as a decoy DNA. Cells were then diluted in OptiMEM 1% FBS and seeded into 96-well plates at 20,000 cells per well and incubated for 20-24 hours. The cells were then treated for 2 hours in the presence of various concentrations of POLQ tracer 9847 and various concentrations of control inhibitor 9617. After incubation, NanoBRET-TE substrate/inhibitor solution was added to a final concentration of 1×, and BRET was measured on a Glomax Discover plate reader.

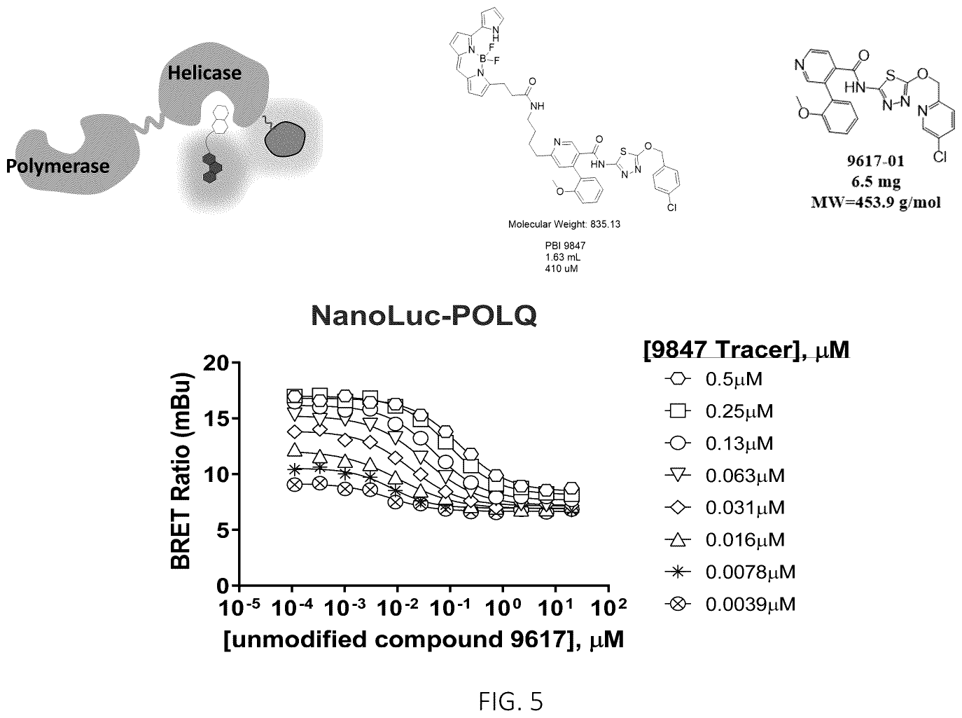

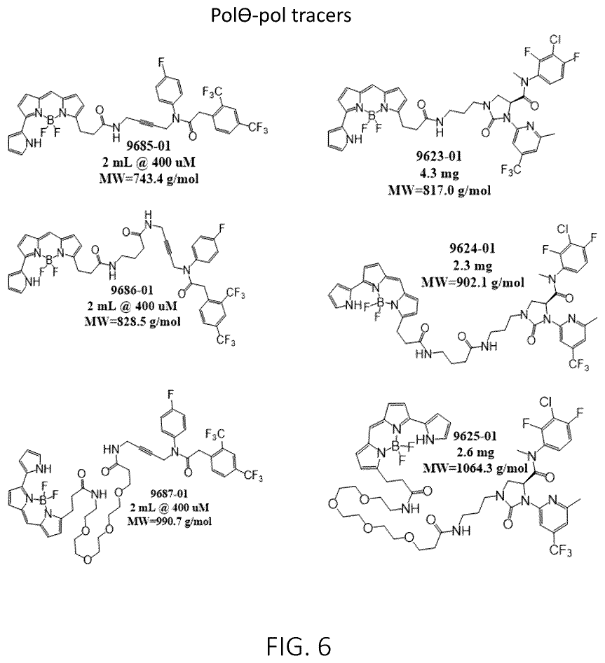

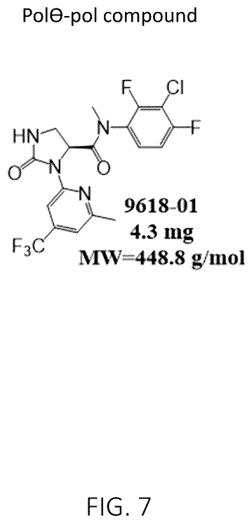

As seen in FIG. 5, a suitable NanoBRET signal with the tracer was observed without added DNA fragments.

### Example 6

### Synthesis of Intermediate 1: (S)-1-(3-aminopropyl)-N-(3-chloro-2,4-difluorophenyl)-N-methyl-3-(6-methyl-4-(trifluoromethyl)pyridin-2-yl)-2-oxoimidazolidine-4-carboxamide

### Step 1: 3-Chloro-2,4-difluoro-N-methylaniline

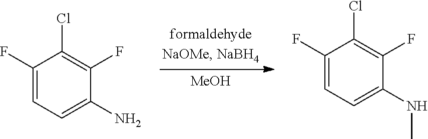

To a 20 mL vial, 3-chloro-2,4-difluoroaniline (1.00 g, 6.11 mmol), 25% w/w NaOMe in MeOH (9.79 mL, 42.8 mmol), and 37% w/w aqueous formaldehyde (0.682 mL, 9.17 mmol) was added. The mixture was stirred for 6 h. To the mixture, NaBH₄ (463 mg, 12.2 mmol) was added. The mixture was stirred at RT for 14 h. The mixture was diluted in water (50 mL) and extracted with DCM (2×50 mL). The organic layer was dried over sodium sulfate, filtered, and the solvents were evaporated. The mixture was purified by silica gel chromatography with 0-50% EtOAc in heptane to afford 3-chloro-2,4-difluoro-N-methylaniline. LRMS [M+H]⁺ 178.

### Step 2: Benzyl (S)-5-((3-chloro-2,4-difluorophenyl)(methyl)carbamoyl)-2-oxoimidazolidine-1-carboxylate

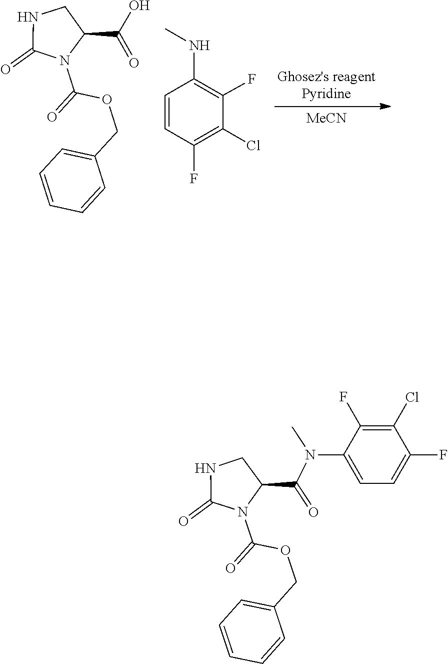

To a 20 mL vial, (S)-3-((benzyloxy)carbonyl)-2-oxoimidazolidine-4-carboxylic acid (375 mg, 1.42 mmol) and dry MeCN (4 mL) was added. To the mixture, Ghosez's reagent (0.375 mL, 2.84 mmol) was added dropwise over 1 min. The mixture was stirred for 30 min at RT. This mixture was added dropwise to a separate mixture 3-chloro-2,4-difluoro-N-methylaniline (630 mg, 3.55 mmol) in dry MeCN (4 mL). After 1 h, to the mixture, pyridine (0.345 mL, 4.26 mmol) was added. The solvents were evaporated, and the residue was purified by silica gel chromatography with 0-10% MeOH in DCM to afford benzyl (S)-5-((3-chloro-2,4-difluorophenyl)(methyl)carbamoyl)-2-oxoimidazolidine-1-carboxylate. LRMS [M+H]⁺ 424.

### Step 3: 3-Benzyl 1-(tert-butyl) (S)-4-((3-chloro-2,4-difluorophenyl)(methyl)carbamoyl)-2-oxoimidazolidine-1,3-dicarboxylate

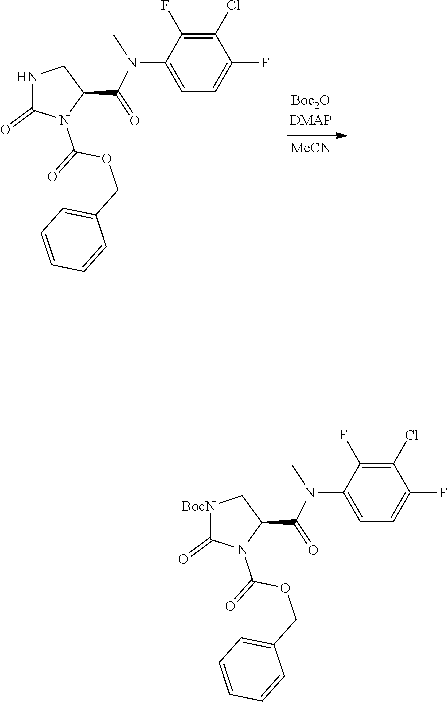 [SMILES: CN(C(=O)[C@@H]1CNC(=O)N1C(=O)OCc1ccccc1)c1ccc(F)c(Cl)c1F ; CN(C(=O)[C@@H]1CN(C(=O)OC(C)(C)C)C(=O)N1C(=O)OCc1ccccc1)c1ccc(F)c(Cl)c1F]

To a 20 mL vial, (S)-5-((3-chloro-2,4-difluorophenyl)(methyl)carbamoyl)-2-oxoimidazolidine-1-carboxylate (599 mg, 1.41 mmol), DMAP (17.3 mg, 0.141 mmol), DCM (4 mL), and Boc₂O (463 mg, 2.12 mmol) was added. The mixture was stirred at RT for 2 h. The mixture was adsorbed to Celite and purified by silica gel chromatography with 0-100% EtOAc in heptane as eluent to afford 3-benzyl 1-(tert-butyl) (S)-4-((3-chloro-2,4-difluorophenyl)(methyl)carbamoyl)-2-oxoimidazolidine-1,3-dicarboxylate. LRMS [M+H-Boc]⁺ 424.

### Step 4: Tert-butyl (S)-4-((3-chloro-2,4-difluorophenyl)(methyl)carbamoyl)-2-oxoimidazolidine-1-carboxylate

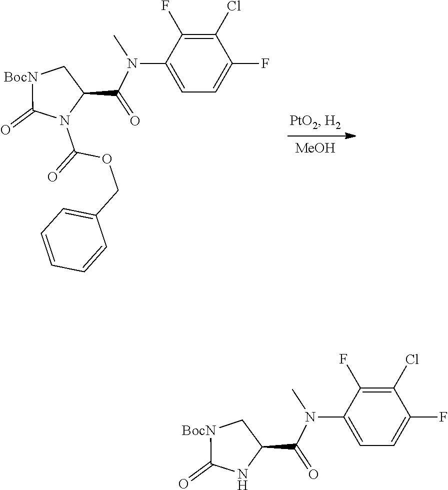 [SMILES: CN(C(=O)[C@@H]1CN(C(=O)OC(C)(C)C)C(=O)N1C(=O)OCc1ccccc1)c1ccc(F)c(Cl)c1F ; CN(C(=O)[C@@H]1CN(C(=O)OC(C)(C)C)C(=O)N1)c1ccc(F)c(Cl)c1F]

To a 20 mL vial, 3-benzyl 1-(tert-butyl) (S)-4-((3-chloro-2,4-difluorophenyl)(methyl)carbamoyl)-2-oxoimidazolidine-1,3-dicarboxylate (741 mg, 1.41 mmol), 10% PtO₂ (321 mg, 0.175 mmol), and MeOH (6 mL) was added. The mixture was placed under an atmosphere of H₂ and stirred for 4 hours. The mixture was filtered through Celite, and the solvents of the filtrate were evaporated to afford tert-butyl (S)-4-((3-chloro-2,4-difluorophenyl)(methyl)carbamoyl)-2-oxoimidazolidine-1-carboxylate. LRMS [M+H]⁺ 390.

### Step 5: Tert-butyl (S)-4-((3-chloro-2,4-difluorophenyl)(methyl)carbamoyl)-3-(6-methyl-4-(trifluoromethyl)pyridin-2-yl)-2-oxoimidazolidine-1-carboxylate

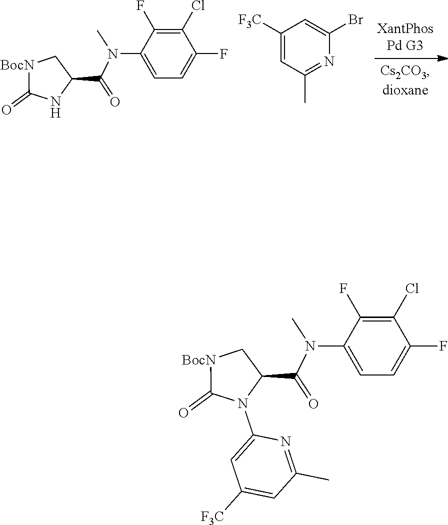 [SMILES: CN(C(=O)[C@@H]1CN(C(=O)OC(C)(C)C)C(=O)N1)c1ccc(F)c(Cl)c1F ; Cc1cc(C(F)(F)F)cc(Br)n1 ; Cc1cc(C(F)(F)F)cc(N2C(=O)N(C(=O)OC(C)(C)C)C[C@H]2C(=O)N(C)c2ccc(F)c(Cl)c2F)n1]

To a 20 mL vial, tert-butyl (S)-4-((3-chloro-2,4-difluorophenyl)(methyl)carbamoyl)-2-oxoimidazolidine-1-carboxylate (50.0 mg, 0.128 mmol), XantPhos Pd G3 (12.4 mg, 0.0128 mmol), Cs₂CO₃ (125.4 mg, 0.384 mmol), and dioxane (1.5 mL) was added. The mixture was sparged with nitrogen for 1 min. The mixture was stirred and heated at 110° C. for 2 h. The mixture was cooled to RT then adsorbed to Celite and purified by silica gel chromatography with 0-50% EtOAc in heptane to afford tert-butyl (S)-4-((3-chloro-2,4-difluorophenyl)(methyl)carbamoyl)-3-(6-methyl-4-(trifluoromethyl)pyridin-2-yl)-2-oxoimidazolidine-1-carboxylate. LRMS [M+H]⁺ 549.

### Step 6: (S)—N-(3-chloro-2,4-difluorophenyl)-N-methyl-3-(6-methyl-4-(trifluoromethyl)pyridin-2-yl)-2-oxoimidazolidine-4-carboxamide

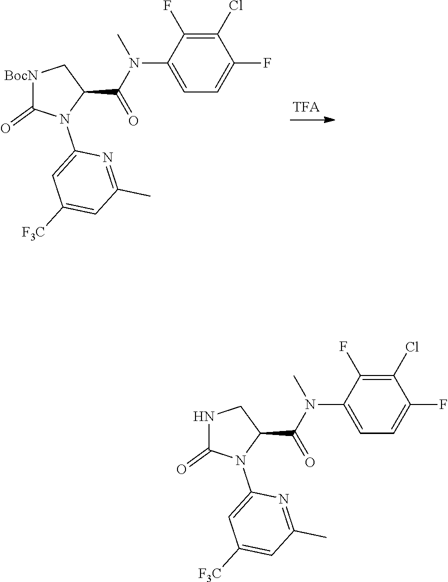 [SMILES: Cc1cc(C(F)(F)F)cc(N2C(=O)N(C(=O)OC(C)(C)C)C[C@H]2C(=O)N(C)c2ccc(F)c(Cl)c2F)n1 ; Cc1cc(C(F)(F)F)cc(N2C(=O)NC[C@H]2C(=O)N(C)c2ccc(F)c(Cl)c2F)n1]

To a 20 mL vial, tert-butyl (S)-4-((3-chloro-2,4-difluorophenyl)(methyl)carbamoyl)-3-(6-methyl-4-(trifluoromethyl)pyridin-2-yl)-2-oxoimidazolidine-1-carboxylate (59.1 mg, 0.108 mmol) and TFA (1 mL) was added. The mixture was stirred for 30 min. The solvents were evaporated, and the residue was purified by silica gel chromatography with 0-100% EtOAc in heptane as eluent to afford (S)—N-(3-chloro-2,4-difluorophenyl)-N-methyl-3-(6-methyl-4-(trifluoromethyl)pyridin-2-yl)-2-oxoimidazolidine-4-carboxamide. LRMS [M+H]⁺ 449.

### Step 7: Tert-butyl (S)-(3-(4-((3-chloro-2,4-difluorophenyl)(methyl)carbamoyl)-3-(6-methyl-4-(trifluoromethyl)pyridin-2-yl)-2-oxoimidazolidin-1-yl)propyl)carbamate

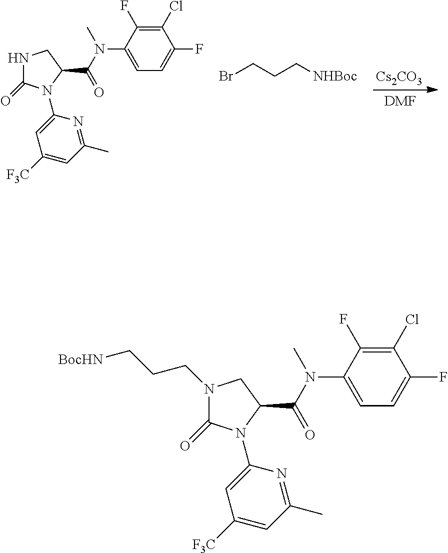

To a 20 mL vial, (S)—N-(3-chloro-2,4-difluorophenyl)-N-methyl-3-(6-methyl-4-(trifluoromethyl)pyridin-2-yl)-2-oxoimidazolidine-4-carboxamide (35.9 mg, 0.0800 mmol), tert-butyl (3-bromopropyl)carbamate (24.8 mg, 0.104 mmol), Cs₂CO₃ (52.1 mg, 0.160 mmol), and DMF (1.5 mL) was added. The mixture was stirred and heated at 80° C. After 4 h, the mixture was diluted in EtOAc and filtered through Celite. The solvents of the filtrate were evaporated. The residue was purified by silica gel chromatography with 0-100% EtOAc in heptane as eluent to afford tert-butyl (S)-(3-(4-((3-chloro-2,4-difluorophenyl)(methyl)carbamoyl)-3-(6-methyl-4-(trifluoromethyl)pyridin-2-yl)-2-oxoimidazolidin-1-yl)propyl)carbamate. LRMS [M+H]⁺ 606.

### Step 8: (S)-1-(3-aminopropyl)-N-(3-chloro-2,4-difluorophenyl)-N-methyl-3-(6-methyl-4-(trifluoromethyl)pyridin-2-yl)-2-oxoimidazolidine-4-carboxamide (Intermediate 1)

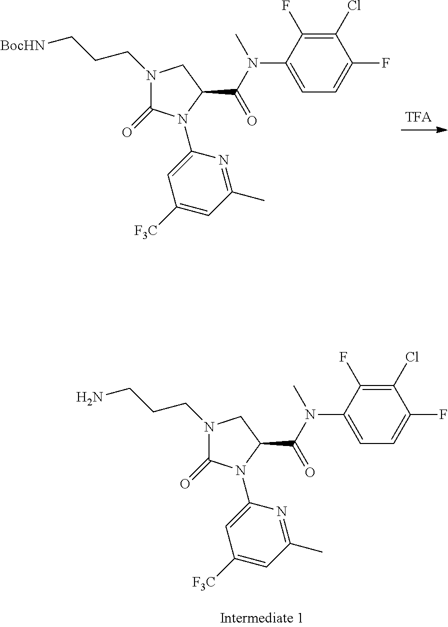 [SMILES: Cc1cc(C(F)(F)F)cc(N2C(=O)N(CCCNC(=O)OC(C)(C)C)C[C@H]2C(=O)N(C)c2ccc(F)c(Cl)c2F)n1 ; Cc1cc(C(F)(F)F)cc(N2C(=O)N(CCCN)C[C@H]2C(=O)N(C)c2ccc(F)c(Cl)c2F)n1]

To a 20 mL vial, tert-butyl (S)-(3-(4-((3-chloro-2,4-difluorophenyl)(methyl)carbamoyl)-3-(6-methyl-4-(trifluoromethyl)pyridin-2-yl)-2-oxoimidazolidin-1-yl)propyl)carbamate (41.2 mg, 0.0680 mmol) and TFA (1 mL) was added. The mixture was stirred for 1 h. The solvents were evaporated to afford (S)-1-(3-aminopropyl)-N-(3-chloro-2,4-difluorophenyl)-N-methyl-3-(6-methyl-4-(trifluoromethyl)pyridin-2-yl)-2-oxoimidazolidine-4-carboxamide (Intermediate 1). LRMS [M+H]⁺ 506.

### Example 7

### Synthesis of Intermediate 2: N-(4-aminobut-2-yn-1-yl)-2-(2,4-bis(trifluoromethyl)phenyl)-N-(4-fluorophenyl)acetamide

### Step 1: 4-Hydroxybut-2-yn-1-yl 4-methylbenzenesulfonate

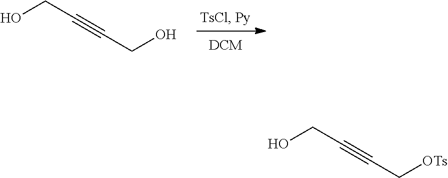 [SMILES: OCC#CCO ; Cc1ccc(S(=O)(=O)OCC#CCO)cc1]

To a 20 mL vial, but-2-yne-1,4-diol (452 mg, 5.25 mmol), DCM (10 mL) and pyridine (0.892 mL, 10.5 mmol) was added. To the mixture, TsCl (500 mg, 2.62 mmol) was added in portions over a min. After stirring at RT for 2 h, the mixture was purified by silica gel chromatography with 0-50% EtOAc in heptane as eluent to afford 4-hydroxybut-2-yn-1-yl 4-methylbenzenesulfonate. LRMS [M+Na]⁺ 263.

### Step 2: Tert-butyl (4-hydroxybut-2-yn-1-yl)carbamate

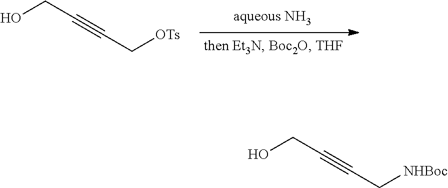 [SMILES: Cc1ccc(S(=O)(=O)OCC#CCO)cc1 ; CC(C)(C)OC(=O)NCC#CCO]

To a 20 mL vial, 4-hydroxybut-2-yn-1-yl 4-methylbenzenesulfonate (37.7 mg, 0.157 mmol) and concentrated aqueous ammonia (1 mL) was added. The mixture was sonicated for 1 min then stirred vigorously for 1 h. The mixture was concentrated, and the residue was coevaporated with toluene twice. The residue was suspended in THF (1 mL) and stirred. To the mixture, Boc₂O (103 mg, 0.471 mmol) followed by Et₃N (0.107 mL, 0.785 mmol) was added. After 30 min, the mixture was adsorbed to Celite and purified by silica gel chromatography with 0-40% EtOAc in heptane to afford tert-butyl (4-hydroxybut-2-yn-1-yl)carbamate. LRMS [M+Na]⁺ 208.

### Step 3: Tert-butyl (4-bromobut-2-yn-1-yl)carbamate

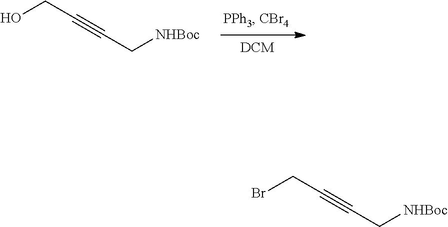

To a 20 mL vial, tert-butyl (4-hydroxybut-2-yn-1-yl)carbamate (17.2 mg, 0.0929 mmol), CBr₄ (61.6 mg, 0.186 mmol), and DCM (1 mL) was added. To the mixture, PPh₃ (48.7 mg, 0.186 mmol) was added. After stirring for 2 h at RT, the mixture was purified by silica gel chromatography with 0-30% EtOAc in heptane as eluent to afford tert-butyl (4-bromobut-2-yn-1-yl)carbamate. LRMS [M+H+MeCN—C₄H₈]⁺ 233.

### Step 4: Tert-butyl (4-((4-fluorophenyl)amino)but-2-yn-1-yl)carbamate

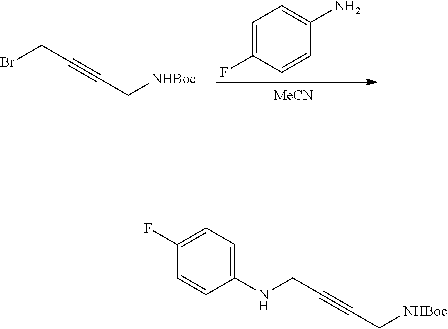 [SMILES: "MeCN" ; CC(C)(C)OC(=O)NCC#CCBr ; Nc1ccc(F)cc1 ; CC(C)(C)OC(=O)NCC#CCNc1ccc(F)cc1]

To a 20 mL vial, tert-butyl (4-bromobut-2-yn-1-yl)carbamate, MeCN (1 mL) and 4-fluoroaniline (0.016 mL, 0.163 mmol) was added. The mixture was heated at 60° C. for 3 h. The mixture was concentrated, and the residue was purified by silica gel chromatography with 0-60% EtOAc in heptane as eluent to afford tert-butyl (4-((4-fluorophenyl)amino)but-2-yn-1-yl)carbamate. LRMS [M+H]⁺ 279.

### Step 5: Tert-butyl (4-(2-(2,4-bis(trifluoromethyl)phenyl)-N-(4-fluorophenyl)acetamido)but-2-yn-1-yl)carbamate

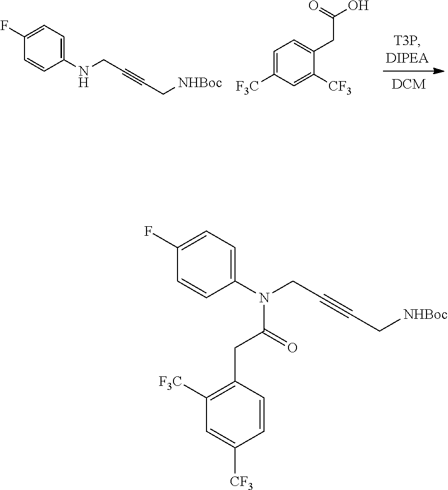 [SMILES: CC(C)(C)OC(=O)NCC#CCNc1ccc(F)cc1 ; O=C(O)Cc1ccc(C(F)(F)F)cc1C(F)(F)F ; CC(C)(C)OC(=O)NCC#CCN(C(=O)Cc1ccc(C(F)(F)F)cc1C(F)(F)F)c1ccc(F)cc1]

To a 20 mL vial, 2-(2,4-bis(trifluoromethyl)phenyl)acetic acid (9.5 mg, 0.035 mmol), tert-butyl (4-((4-fluorophenyl)amino)but-2-yn-1-yl)carbamate (6.5 mg, 0.023 mmol), DIPEA (0.016 mL, 0.093 mmol), and DCM (1 mL) was added. To the mixture, T3P (50% w/w in EtOAc, 0.029 mL, 0.047 mmol) was added. The mixture was stirred for 10 min at RT. The mixture was purified by silica gel chromatography with 0-60% EtOAc in heptane as eluent to afford tert-butyl (4-(2-(2,4-bis(trifluoromethyl)phenyl)-N-(4-fluorophenyl)acetamido)but-2-yn-1-yl)carbamate. LRMS [M−H]⁻ 531.

### Step 6: N-(4-aminobut-2-yn-1-yl)-2-(2,4-bis(trifluoromethyl)phenyl)-N-(4-fluorophenyl)acetamide (Intermediate 2)

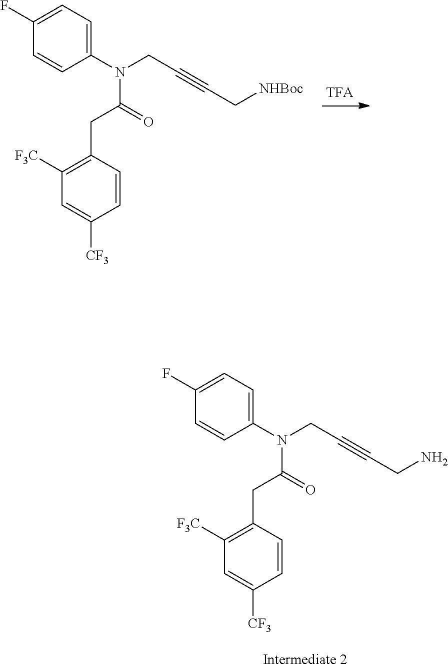 [SMILES: CC(C)(C)OC(=O)NCC#CCN(C(=O)Cc1ccc(C(F)(F)F)cc1C(F)(F)F)c1ccc(F)cc1 ; NCC#CCN(C(=O)Cc1ccc(C(F)(F)F)cc1C(F)(F)F)c1ccc(F)cc1]

To a 20 mL vial, tert-butyl (4-(2-(2,4-bis(trifluoromethyl)phenyl)-N-(4-fluorophenyl)acetamido)but-2-yn-1-yl)carbamate (10.0 mg, 0.0188 mmol) and TFA (1 mL) was added. The mixture was stirred for 10 min. The solvents were evaporated to afford N-(4-aminobut-2-yn-1-yl)-2-(2,4-bis(trifluoromethyl)phenyl)-N-(4-fluorophenyl)acetamide (Intermediate 2). LRMS [M+H]⁺ 433.

### Example 8

### Synthesis of Intermediate 3: 6-Chloro-4-(2-methoxyphenyl)nicotinic Acid

### Step 1: 6-Chloro-4-(2-methoxyphenyl)nicotinate

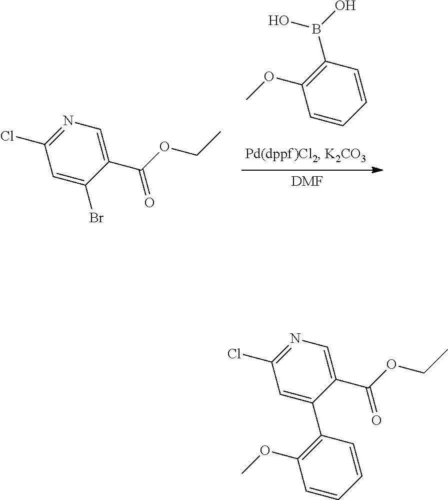

To a 100 mL flask, ethyl 4-bromo-6-chloronicotinate (1.00 g, 3.80 mmol), (2-methoxyphenyl)boronic acid (689 mg, 4.50 mmol), K₂CO₃ (3.10 g, 22.7 mmol), Pd(dppf)Cl₂ (218 mg, 0.200 mmol), and DMF (12 mL) was added. The mixture was stirred at 100° C. for 14 h. The reaction was cooled to room temperature, diluted with water, and extracted with EtOAc. The organic layer was dried with Na₂SO₄, filtered, and the solvents were evaporated. The crude product was purified by silica gel chromatography with 0-30% EtOAc in heptane as eluent to afford ethyl 6-chloro-4-(2-methoxyphenyl)nicotinate. LRMS [M+H]⁺: 292.

### Step 2: 6-Chloro-4-(2-methoxyphenyl)nicotinic Acid

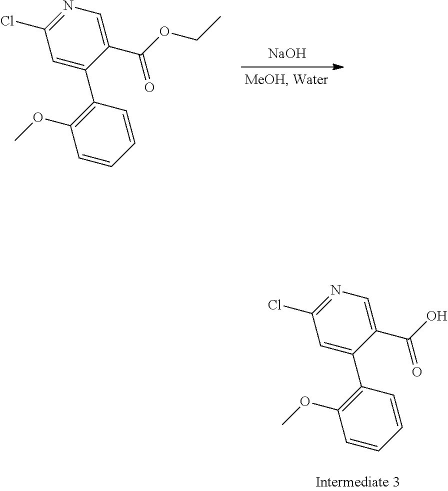 [SMILES: CCOC(=O)c1cnc(Cl)cc1-c1ccccc1OC ; "NaOH" ; COc1ccccc1-c1cc(Cl)ncc1C(=O)O]

To a 20 mL vial, ethyl 6-chloro-4-(2-methoxyphenyl)nicotinate (858 mg, 2.94 mmol) and MeOH (6 mL) was added. To the mixture, 2 M aq NaOH (6 mL) was added. The mixture was stirred and heated at 70° C. for 30 min. The pH of the mixture was adjusted to 7 with 1 M aq HCl. The mixture was extracted with EtOAc, the organic layer dried with Na₂SO₄, filtered, and the solvents of the filtrate were evaporated. The crude product was purified by silica gel chromatography with 0-100% EtOAc in heptane as eluent to afford Intermediate 3. LRMS [M+H]⁺: 264.

### Example 9

### Synthesis of Intermediate 4: 5-((4-chlorobenzyl)oxy)-1,3,4-thiadiazol-2-amine

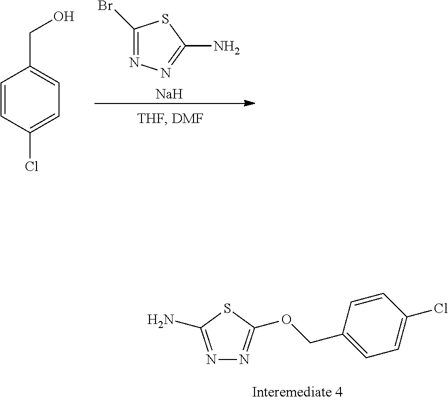

To a 100 mL flask, (4-chlorophenyl)methanol (1.25 g, 8.80 mmol) and anhydrous THF (12 mL) was added. The mixture was stirred at 0° C. To the mixture, NaH (316 mg, 13.2 mmol) as a suspension in anhydrous THF (6 mL) was added dropwise. After 10 min, to the mixture, 5-bromo-1,3,4-thiadiazol-2-amine (1.58 g, 8.80 mmol) as a solution in anhydrous DMF (6 mL) was added. The mixture was stirred for 1 h. The mixture was quenched with water then extracted with EtOAc, the organic layer dried with Na₂SO₄, filtered, and the solvents of the filtrate were evaporated. The crude product was purified by silica gel chromatography with 0-100% EtOAc in heptane as eluent to afford Intermediate 4. LRMS [M+H]⁺: 242.

### Example 10

### Synthesis of Intermediate 5: 6-(4-aminobutyl)-N-(5-((4-chlorobenzyl)oxy)-1,3,4-thiadiazol-2-yl)-4-(2-methoxyphenyl)nicotinamide

### Step 1: 6-chloro-N-(5-((4-chlorobenzyl)oxy)-1,3,4-thiadiazol-2-yl)-4-(2-methoxyphenyl)nicotinamide

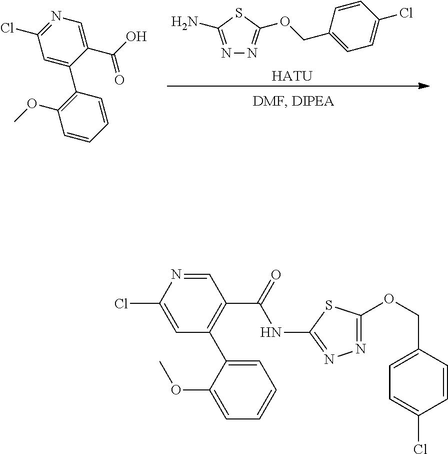

To a 20 mL vial, Intermediate 3 (615 mg, 2.30 mmol), HATU (1.33 g, 3.50 mmol), DIPEA (1.22 mL, 7.00 mmol), and DMF (6 mL) was added. The mixture was stirred at RT for 10 min. To the mixture, Intermediate 4 (733 mg, 3.00 mmol) as a solution in DMF (6 mL) was added. The mixture was stirred at RT for 30 min. The mixture was diluted with DMF and purified by reversed phase HPLC (MeCN/water w/ 0.1% TFA) to afford 6-chloro-N-(5-((4-chlorobenzyl)oxy)-1,3,4-thiadiazol-2-yl)-4-(2-methoxyphenyl)nicotinamide. LRMS [M+H]⁺: 487.

### Step 2: N-(5-((4-chlorobenzyl)oxy)-1,3,4-thiadiazol-2-yl)-6-(4-(1,3-dioxoisoindolin-2-yl)butyl)-4-(2-methoxyphenyl)nicotinamide

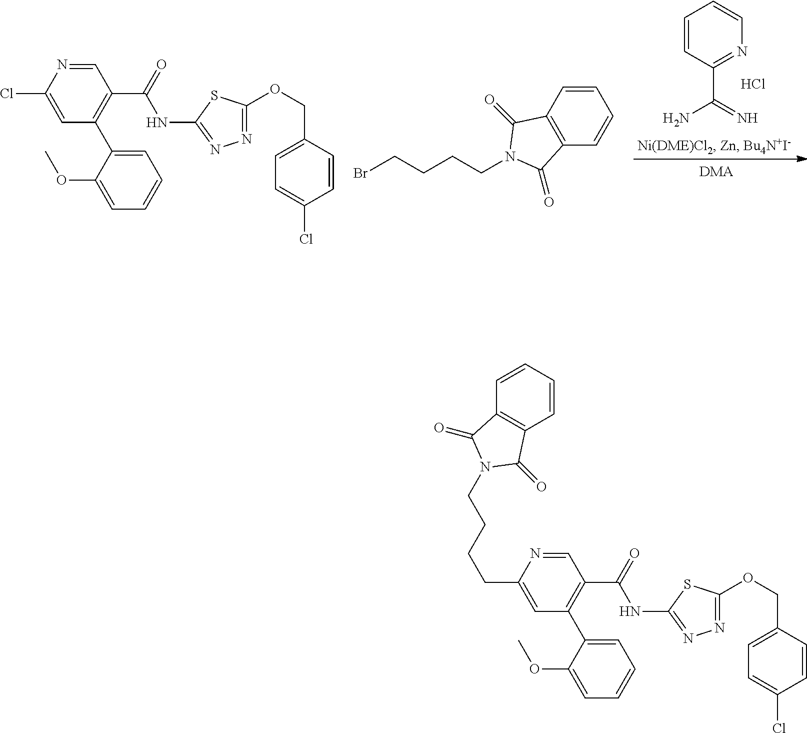

To a 20 mL vial, 6-chloro-N-(5-((4-chlorobenzyl)oxy)-1,3,4-thiadiazol-2-yl)-4-(2-methoxyphenyl)nicotinamide (165 mg, 0.340 mmol) and DMA (3 mL) was added. To the mixture, 2-(4-bromobutyl)isoindoline-1,3-dione (143 mg, 0.510 mmol), Zn powder (44.3 mg, 0.680 mmol), Ni(DME)Cl₂ (37.2 mg, 0.170 mmol), picolinimidamide hydrochloride (26.7 mg, 0.170 mmol), and tetrabutylammonium iodide (250 mg, 0.680 mmol) were added. The mixture was degassed with nitrogen, and the mixture was stirred at RT for 14 h. The mixture was diluted with EtOAc and filtered through Celite. The filtrate was washed 3 times with water, and the organic layer was dried with Na₂SO₄, filtered, and the solvents of the filtrate were evaporated. The crude product was purified by silica gel chromatography with 0-100% EtOAc in heptane as eluent to afford N-(5-((4-chlorobenzyl)oxy)-1,3,4-thiadiazol-2-yl)-6-(4-(1,3-dioxoisoindolin-2-yl)butyl)-4-(2-methoxyphenyl)nicotinamide. LRMS [M+H]⁺: 654.

### Step 3: 6-(4-aminobutyl)-N-(5-((4-chlorobenzyl)oxy)-1,3,4-thiadiazol-2-yl)-4-(2-methoxyphenyl)nicotinamide

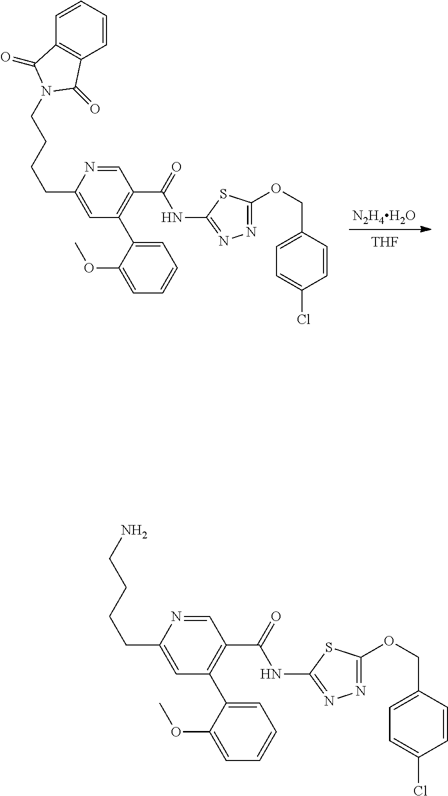

To a 20 mL vial, N-(5-((4-chlorobenzyl)oxy)-1,3,4-thiadiazol-2-yl)-6-(4-(1,3-dioxoisoindolin-2-yl)butyl)-4-(2-methoxyphenyl)nicotinamide (30.2 mg, 0.0500 mmol) and anhydrous THF (5 mL) was added. To the mixture, hydrazine monohydrate (30 uL, 1.0 mmol) was added. The mixture was stirred and heated at 50° C. The mixture was concentrated then coevaporated with toluene. The crude product was purified by reversed phase HPLC (MeCN/water w/ 0.05 M TEAB) to afford Intermediate 5 LRMS [M+H]⁺: 524.

### Example 11

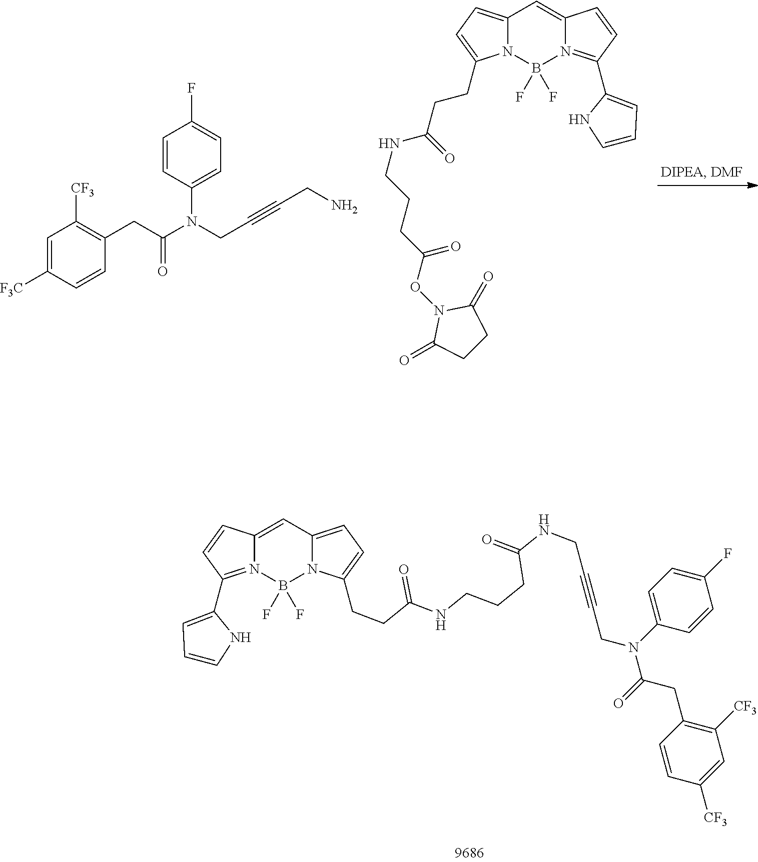

To a 20 mL vial, Intermediate 2 (2.7 mg, 0.0063 mmol), NanoBRET™ 590 C4 succinimdyl ester (3.5 mg, 0.0069 mmol), DMF (1 mL), and DIPEA (0.011 mL, 0.062 mmol) was added. The mixture was stirred for 2 h at RT. The mixture was purified by prep HPLC (MeCN/water, w/ 0.1% TFA) to afford 9686. LRMS [M+H]⁺ 829. ¹H NMR (400 MHz, DMSO-d₆) δ 11.42 (d, J=4.2 Hz, 1H), 8.21 (t, J=5.6 Hz, 1H), 8.04 (d, J=8.0 Hz, 1H), 7.96 (d, J=6.0 Hz, 1H), 7.94 (s, 1H), 7.70 (d, J=8.1 Hz, 1H), 7.49 (dd, J=8.7, 5.0 Hz, 2H), 7.44 (s, 1H), 7.42-7.32 (m, 4H), 7.27 (d, J=3.1 Hz, 1H), 7.17 (d, J=4.5 Hz, 1H), 7.01 (d, J=4.0 Hz, 1H), 6.34 (t, J=4.3 Hz, 2H), 4.45 (s, 2H), 3.84 (d, J=5.2 Hz, 2H), 3.66 (s, 2H), 3.14 (t, J=7.8 Hz, 2H), 3.06 (q, J=6.6 Hz, 2H), 2.09 (t, J=7.6 Hz, 2H), 1.63 (p, J=7.4 Hz, 2H).
The synthesis of the compounds in the following table were conducted in analogy to that of 9686 from the corresponding intermediate and commercially available NanoBRET™ 590 succinimdyl ester.

| Compound | Structure | LRMS [M + H]⁺ |
|---|---|---|
| 9623 | 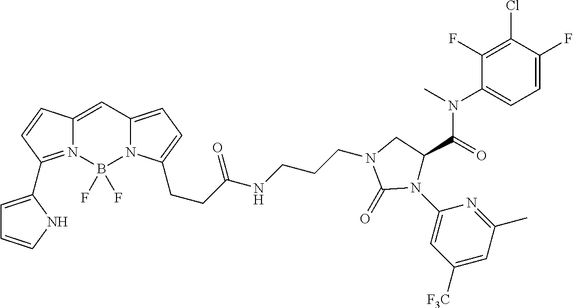 | 817 |
| 9624 | 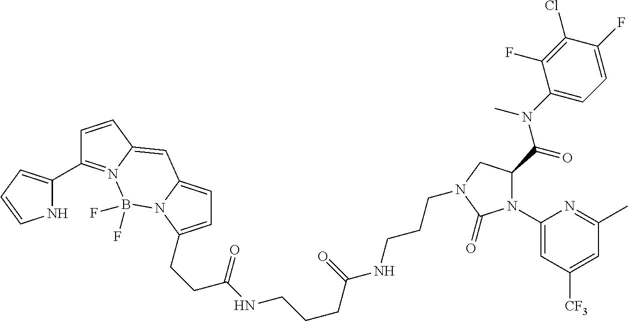 | 902 |
| 9625 | 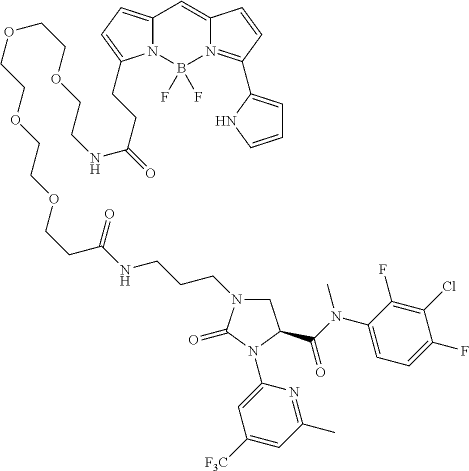 | 1064 |
| 9685 | 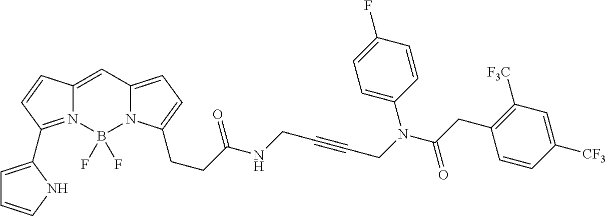 | 744 |
| 9687 | 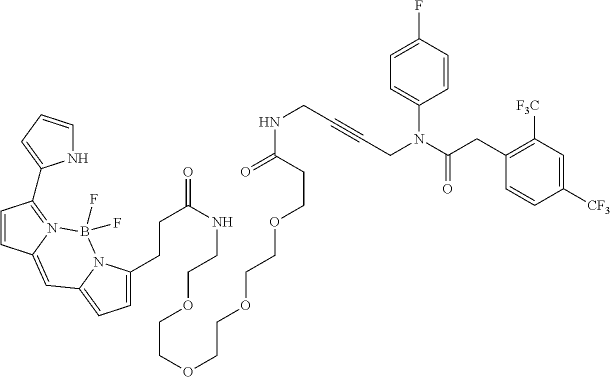 | 991 |
| 9847 | 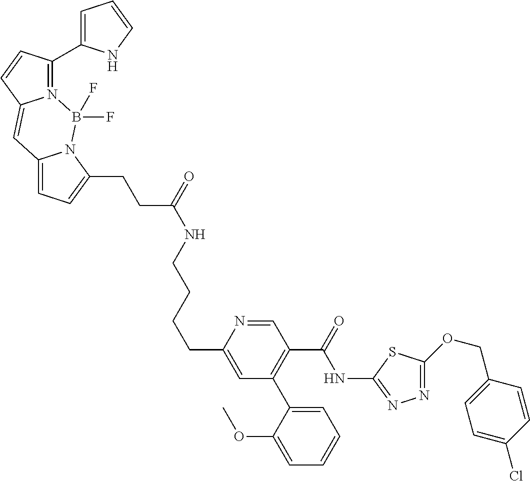 | 835 |
| 9848 | 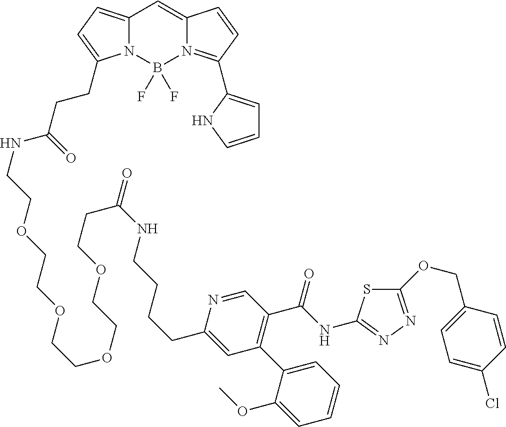 | 920 |
| 9849 | 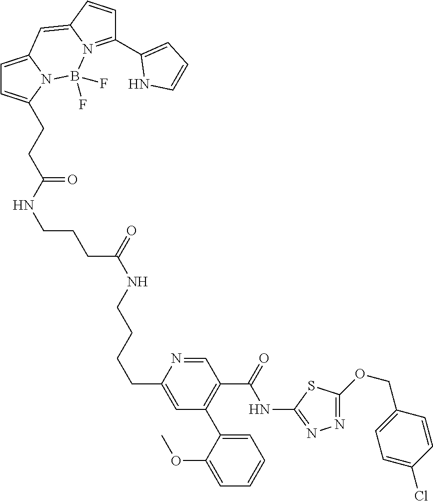 | 1082 |

### Example 12

### Enhancement of Target Engagement at Polymerase Domain of Polymerase Theta in Live Cells—Chemical Delivery of Damaged DNA

During development of the target engagement assay for Polymerase Theta (POL Theta; PolΘ) described herein, experiments were conducted to evaluate live cell target engagement via ectopic delivery of DNA fragments. HEK293 cells with a stably integrated POLQ-NanoLuc (Nluc) fusion were transfected with different amounts of stubby oligo DNA fragments using Minus TransIT-X2, and the cells distributed into wells of 96-well plates at 20,000-40,000 cells per well. 4-6 hours post transfection, cells were treated for 2 hours in the presence of 1 uM POLQ tracer 9686 was added to all wells, and 10 uM control inhibitor 9618 or DMSO (control) was added. After incubation, NanoBRET-TE substrate/inhibitor solution was added to a final concentration of 1×, and BRET was measured on a Glomax Discover plate reader.

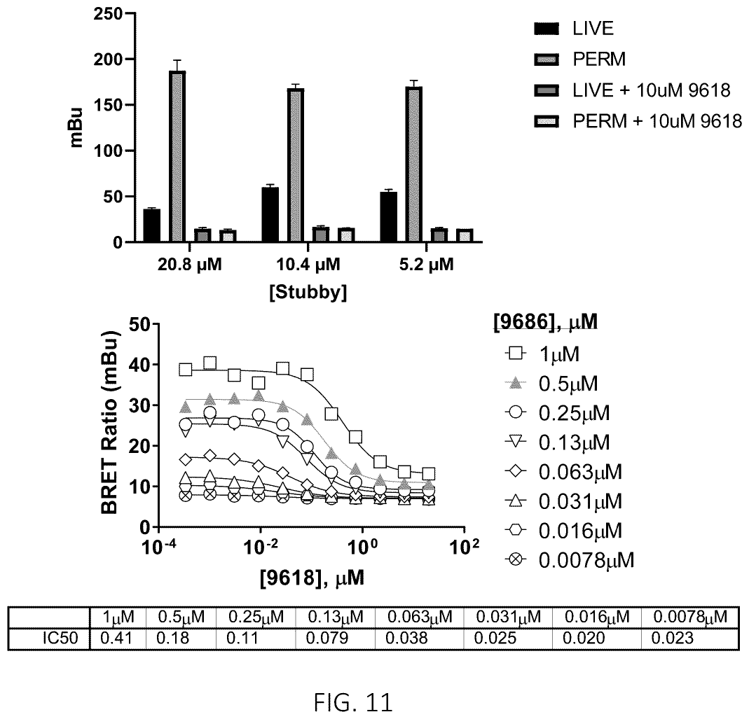

As shown in FIG. 11, in each case, addition of DNA fragments potentiated the NanoBRET signal with the tracer. Enhanced target engagement of unmodified drug was also observed in the presence of DNA fragment. Specifically, the BRET signal is reduced with increasing concentrations of the polymerase theta inhibitor 9618 when DNA fragments are present, indicating high affinity of the tracer 9686 with polymerase theta and enhanced target engagement of the inhibitor with polymerase theta when coordinative binding of DNA fragments occurs.

1. A tracer compound of formula (Ia) or (Ib): 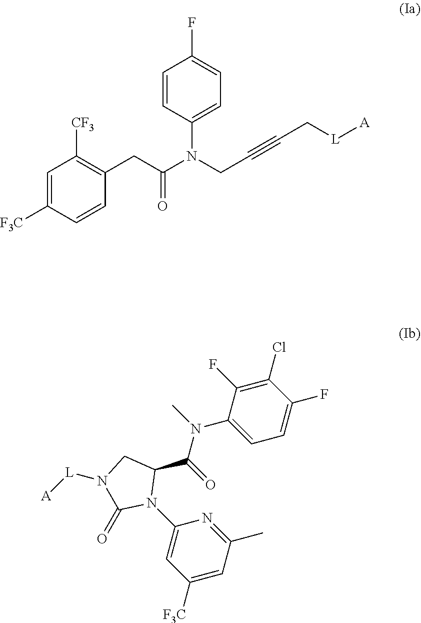
or a salt thereof, wherein:
L is a linker; and
A is an energy transfer acceptor.

2. The tracer compound of claim 1, or a salt thereof, wherein L has a formula:
—NH—(CH₂CH₂O)ₙ₁—(CH₂)ₙ₂—(C(O)NH)ₙ₃—(CH₂)ₙ₄—
wherein n1, n2, and n4 are each independently selected from 0, 1, 2, 3, 4, 5, and 6, and n3 is 0 or 1.

3. The tracer compound of claim 1, or a salt thereof, wherein L has a formula:

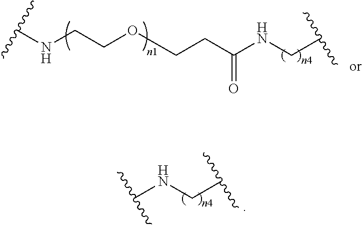

4. The tracer compound of claim 1, or a salt thereof, wherein A is a fluorophore selected from a xanthene, a cyanine, a naphthalene, an oxadiazole, a pyrene, an oxazine, an acridine, an arylmethine, a tetrapyrrole, and a boron dipyrromethene.

5. The tracer compound of claim 4, or a salt thereof, wherein A is a BODIPY dye.

6. The tracer compound of claim 5, or a salt thereof, wherein A has a formula:

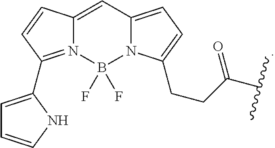

7. The tracer compound of claim 1, selected from:

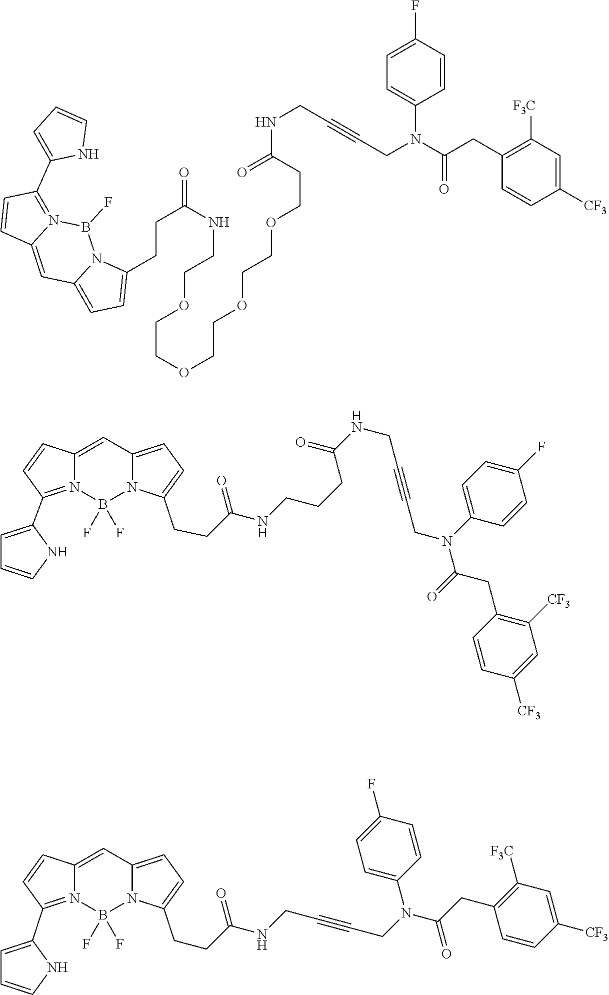

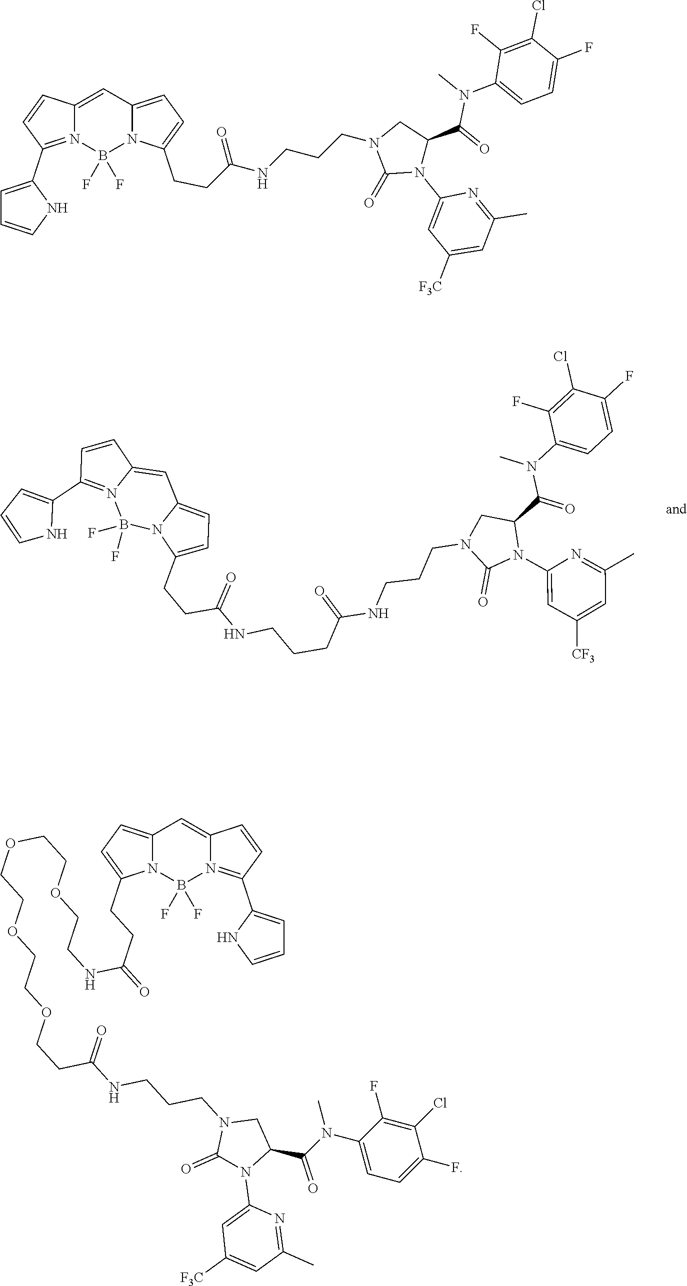

8. A tracer compound of formula (II): 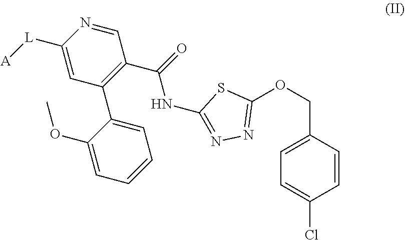
or a salt thereof, wherein:
L is a linker; and
A is an energy transfer acceptor.

9. The tracer compound of claim 8, or a salt thereof, wherein L has a formula:
—NH—(CH₂CH₂O)ₙ₁—(CH₂)ₙ₂—(C(O)NH)ₙ₃—(CH₂)ₙ₄—
wherein n1, n2, and n4 are each independently selected from 0, 1, 2, 3, 4, 5, and 6, and n3 is 0 or 1.

10. The tracer compound of claim 8, or a salt thereof, wherein L has a formula:

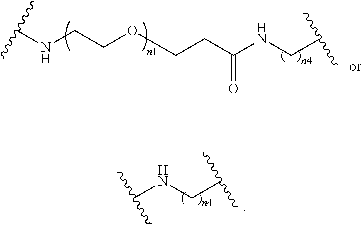

11. The tracer compound of claim 8, or a salt thereof, wherein A is a fluorophore selected from a xanthene, a cyanine, a naphthalene, an oxadiazole, a pyrene, an oxazine, an acridine, an arylmethine, a tetrapyrrole, and a boron dipyrromethene.

12. The tracer compound of claim 11, or a salt thereof, wherein A is a BODIPY dye.

13. The tracer compound of claim 12, or a salt thereof, wherein A has a formula:

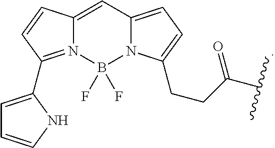

14. The tracer compound of claim 8, selected from:

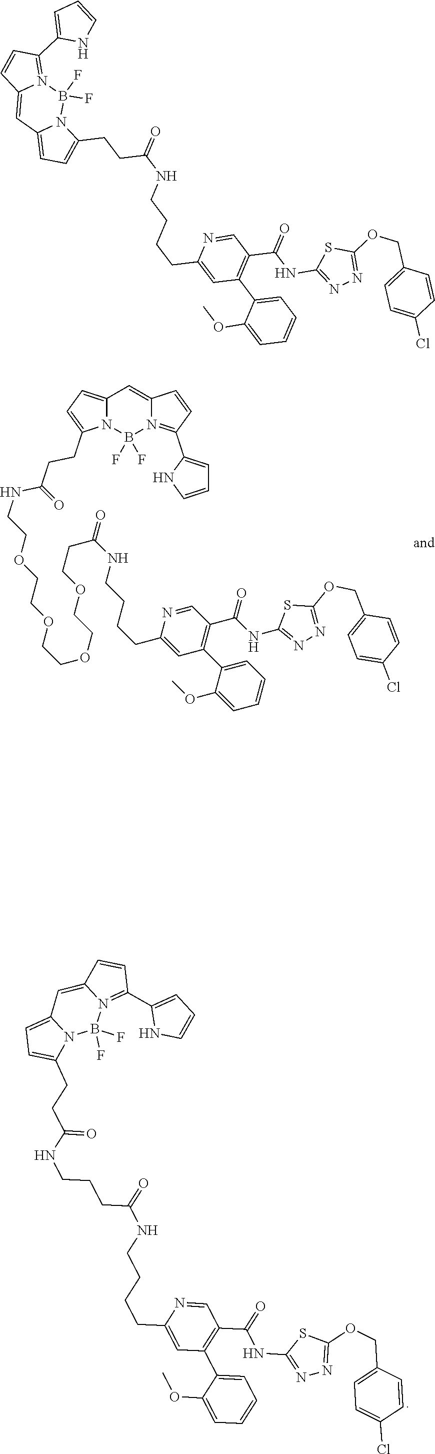

15. A fusion protein comprising a bioluminescent reporter and DNA polymerase theta (Polθ).

16. The fusion protein of claim 15, wherein the bioluminescent reporter comprises a luciferase.

17. (canceled)

18. A system comprising:
a) a cell that expresses a fusion protein comprising a bioluminescent reporter and DNA polymerase theta (Polθ);
b) one or more DNA fragments;
c) a tracer compound of claim 4, wherein the tracer compound binds coordinatively with the one or more DNA fragments to the polymerase domain of Polθ; and
d) a substrate for the bioluminescent reporter.

19-21. (canceled)

22. A system comprising:
a) a cell that expresses a fusion protein comprising a bioluminescent reporter and DNA polymerase theta (Polθ);
b) a tracer compound of claim 11, wherein the tracer compound binds to the helicase domain of Polθ; and
c) a substrate for the bioluminescent reporter.

23-31. (canceled)

32. A method of detecting an interaction between a test compound and DNA polymerase theta (Polθ), the method comprising:
a) providing a cell that expresses a fusion protein comprising a bioluminescent reporter and DNA polymerase theta (Polθ);
b) contacting the cell with one or more DNA fragments, a substrate for the bioluminescent reporter, and a tracer compound of claim 4, wherein the tracer compound binds coordinatively with the one or more DNA fragments to Polθ;
c) detecting a first BRET signal, wherein the first BRET signal is indicative of coordinative binding of the tracer compound and the one or more DNA fragments to Polθ;
d) contacting the cell with a test compound; and
e) detecting a second BRET signal,
wherein a decrease in the second BRET signal from the first BRET signal indicates that the test compound is an engager of Polθ.

33.-34. (canceled)

35. A method of detecting an interaction between a test compound and DNA polymerase theta (Polθ), the method comprising:
a) providing a cell that expresses a fusion protein comprising a bioluminescent reporter and DNA polymerase theta (Polθ);
b) contacting the cell with a substrate for the bioluminescent reporter, and a tracer compound of claim 11, wherein the tracer compound binds to Polθ;
c) detecting a first BRET signal, wherein the first BRET signal is indicative of binding of the tracer compound to Polθ;
d) contacting the cell with a test compound; and
e) detecting a second BRET signal,
wherein a decrease in the second BRET signal from the first BRET signal indicates that the test compound is an engager of Polθ.

36.-40. (canceled)


SEQUENCE LISTING (4 sequences)

SEQ ID NO: 1 - protein, 169 aa, Oplophorus gracilirostris
FTLADFVGDWQQTAGYNQDQVLEQGGLSSLFQALGVSVTPIQKVVLSGENGLKADIHVIIPYEGLSGFQMGLIEMIFKVVYPVDDHHFKIILHYGTLVIDGVTPNMIDYFGRPYPGIAVFDGKQITVTGTLWNGNKIYDERLINPDGSLLFRVTINGVTGWRLCENILA

SEQ ID NO: 2 - protein, 2590 aa, Homo sapiens
MNLLRRSGKRRRSESGSDSFSGSGGDSSASPQFLSGSVLSPPPGLGRCLKAAAAGECKPTVPDYERDKLLLANWGLPKAVLEKYHSFGVKKMFEWQAECLLLGQVLEGKNLVYSAPTSAGKTLVAELLILKRVLEMRKKALFILPFVSVAKEKKYYLQSLFQEVGIKVDGYMGSTSPSRHFSSLDIAVCTIERANGLINRLIEENKMDLLGMVVVDELHMLGDSHRGYLLELLLTKICYITRKSASCQADLASSLSNAVQIVGMSATLPNLELVASWLNAELYHTDFRPVPLLESVKVGNSIYDSSMKLVREFEPMLQVKGDEDHVVSLCYETICDNHSVLLFCPSKKWCEKLADIIAREFYNLHHQAEGLVKPSECPPVILEQKELLEVMDQLRRLPSGLDSVLQKTVPWGVAFHHAGLTFEERDIIEGAFRQGLIRVLAATSTLSSGVNLPARRVIIRTPIFGGRPLDILTYKQMVGRAGRKGVDTVGESILICKNSEKSKGIALLQGSLKPVRSCLQRREGEEVTGSMIRAILEIIVGGVASTSQDMHTYAACTFLAASMKEGKQGIQRNQESVQLGAIEACVMWLLENEFIQSTEASDGTEGKVYHPTHLGSATLSSSLSPADTLDIFADLQRAMKGFVLENDLHILYLVTPMFEDWTTIDWYRFFCLWEKLPTSMKRVAELVGVEEGFLARCVKGKVVARTERQHRQMAIHKRFFTSLVLLDLISEVPLREINQKYGCNRGQIQSLQQSAAVYAGMITVFSNRLGWHNMELLLSQFQKRLTFGIQRELCDLVRVSLLNAQRARVLYASGFHTVADLARANIVEVEVILKNAVPFKSARKAVDEEEEAVEERRNMRTIWVTGRKGLTEREAAALIVEEARMILQQDLVEMGVQWNPCALLHSSTCSLTHSESEVKEHTFISQTKSSYKKLTSKNKSNTIFSDSYIKHSPNIVQDLNKSREHTSSFNCNFQNGNQEHQTCSIFRARKRASLDINKEKPGASQNEGKTSDKKVVQTFSQKTKKAPLNFNSEKMSRSFRSWKRRKHLKRSRDSSPLKDSGACRIHLQGQTLSNPSLCEDPFTLDEKKTEFRNSGPFAKNVSLSGKEKDNKTSFPLQIKQNCSWNITLTNDNFVEHIVTGSQSKNVTCQATSVVSEKGRGVAVEAEKINEVLIQNGSKNQNVYMKHHDIHPINQYLRKQSHEQTSTITKQKNIIERQMPCEAVSSYINRDSNVTINCERIKLNTEENKPSHFQALGDDISRTVIPSEVLPSAGAFSKSEGQHENFLNISRLQEKTGTYTTNKTKNNHVSDLGLVLCDFEDSFYLDTQSEKIIQQMATENAKLGAKDTNLAAGIMQKSLVQQNSMNSFQKECHIPFPAEQHPLGATKIDHLDLKTVGTMKQSSDSHGVDILTPESPIFHSPILLEENGLFLKKNEVSVTDSQLNSFLQGYQTQETVKPVILLIPQKRTPTGVEGECLPVPETSLNMSDSLLFDSFSDDYLVKEQLPDMQMKEPLPSEVTSNHFSDSLCLQEDLIKKSNVNENQDTHQQLTCSNDESIIFSEMDSVQMVEALDNVDIFPVQEKNHTVVSPRALELSDPVLDEHHQGDQDGGDQDERAEKSKLTGTRQNHSFIWSGASFDLSPGLQRILDKVSSPLENEKLKSMTINFSSLNRKNTELNEEQEVISNLETKQVQGISFSSNNEVKSKIEMLENNANHDETSSLLPRKESNIVDDNGLIPPTPIPTSASKLTFPGILETPVNPWKTNNVLQPGESYLFGSPSDIKNHDLSPGSRNGFKDNSPISDTSFSLQLSQDGLQLTPASSSSESLSIIDVASDQNLFQTFIKEWRCKKRFSISLACEKIRSLTSSKTATIGSRFKQASSPQEIPIRDDGFPIKGCDDTLVVGLAVCWGGRDAYYFSLQKEQKHSEISASLVPPSLDPSLTLKDRMWYLQSCLRKESDKECSVVIYDFIQSYKILLLSCGISLEQSYEDPKVACWLLDPDSQEPTLHSIVTSFLPHELPLLEGMETSQGIQSLGLNAGSEHSGRYRASVESILIFNSMNQLNSLLQKENLQDVFRKVEMPSQYCLALLELNGIGFSTAECESQKHIMQAKLDAIETQAYQLAGHSFSFTSSDDIAEVLFLELKLPPNREMKNQGSKKTLGSTRRGIDNGRKLRLGRQFSTSKDVLNKLKALHPLPGLILEWRRITNAITKVVFPLQREKCLNPFLGMERIYPVSQSHTATGRITFTEPNIQNVPRDFEIKMPTLVGESPPSQAVGKGLLPMGRGKYKKGFSVNPRCQAQMEERAADRGMPFSISMRHAFVPFPGGSILAADYSQLELRILAHLSHDRRLIQVLNTGADVFRSIAAEWKMIEPESVGDDLRQQAKQICYGIIYGMGAKSLGEQMGIKENDAACYIDSFKSRYTGINQFMTETVKNCKRDGFVQTILGRRRYLPGIKDNNPYRKAHAERQAINTIVQGSAADIVKIATVNIQKQLETFHSTFKSHGHREGMLQSDQTGLSRKRKLQGMFCPIRGGFFILQLHDELLYEVAEEDVVQVAQIVKNEMESAVKLSVKLKVKVKIGASWGELKDFDV

SEQ ID NO: 3 - DNA, 59 bp, Synthetic construct
  source 1..59: mol_type = other DNA
tagcgaaggatgtgaacctaatccctgctcccgcggccgatctgccggccgcgggagca

SEQ ID NO: 4 - DNA, 43 bp, Synthetic construct
  source 1..43: mol_type = other DNA
cctaatccctgctcccgcggccgatctgcccgccgcgggagca